(19) United States
(12) Patent Application Publication
(10) Pub. No.: US 2026/0308218 A1
(43) Pub. Date: Oct. 8, 2026

(54) FUSION PROTEINS OF IL-24 AND IMMUNOSTIMULATORY CYTOKINES, NK CELLS EXPRESSING IL-24 OR FUSION PROTEINS, AND METHODS OF USE THEREOF

(71) Applicant: Virginia Commonwealth University, Richmond, VA (US)

(72) Inventors: Paul B. Fisher, Richmond, VA (US); Swadesh Das, Richmond, VA (US); Amit Kumar, Richmond, VA (US); Luni Emdad, Richmond, VA (US)

(21) Appl. No.: 19/483,519

(22) PCT Filed: May 10, 2024
(86) PCT No.: PCT/US2024/028836

§ 371 Date: Nov. 11, 2025

Related U.S. Application Data
(60) Provisional application No. 63/501,661, filed on May 11, 2023.

(51) Int. Cl. C07K 14/54; A61K 38/00; A61K 41/00; A61K 48/00; A61P 35/00; C07K 14/55; C07K 14/62; C07K 14/705; C12N 15/86

(52) U.S. Cl. CPC C07K 14/54; A61K 41/0028; A61K 48/0033; A61K 48/005; A61P 35/00; C07K 14/5434; C07K 14/5443; C07K 14/55; C07K 14/62; C07K 14/7051; C12N 15/86; A61K 38/00; C07K 2319/02; C12N 2710/10043

(57) ABSTRACT

The present disclosure provides fusion proteins of IL-24 with immunostimulatory cytokines and natural killer (NK) cells genetically engineered to express IL-24 or such fusion proteins, and methods of using these proteins and cells.

### RELATED APPLICATIONS

This application claims the benefit of and priority to U.S. Provisional Application No. 63/501,661 filed May 11, 2023, the entire disclosure of which is hereby incorporated by reference in its entirety for all purposes.

### FIELD OF THE INVENTION

The present disclosure relates generally to fusion proteins of Interleukin 24 (IL-24), a.k.a. Melanoma Differentiation Associated Gene-7 (MDA-7), with immunostimulatory cytokines. The present disclosure also relates to natural killer (NK) cells genetically engineered to express IL-24 or such fusion proteins. Methods of using these proteins and cells are also provided herein.

### BACKGROUND

IL-24, a secreted protein of the IL-10 family, functions as a cytokine at normal physiological levels and is expressed in tissues of the immune system. IL-24 plays a prominent role in inhibiting tumor growth, invasion, metastasis and angiogenesis and has been reported to target tumor stem/initiating cells for death. IL-24 can selectively induce cell death in cancer cells without affecting normal cells. Adenovirus that replicates selectively in cancer cells and expresses IL-24 has been shown to substantially inhibit tumor progress in animal models.

Although significant developments have been made, there remains a need for new and useful IL-24-based cancer therapies.

### SUMMARY OF THE INVENTION

The present disclosure is based, in part, upon the strategy and implementation of a therapeutic fusion protein including IL-24 and an immunostimulatory cytokine (e.g., IL-15, IL-2, or IL-12). Such therapeutic fusion protein retains the cancer-selective cytotoxicity of IL-24 and stimulates proliferation and activation of a wide range of immune cells, such as NK cells and T cells, resulting enhanced immune responses to tumors. Furthermore, the fusion protein can maintain the low toxicity of IL-24 and certain immunostimulatory cytokines, such as IL-15, to achieve a favorable safety profile. In addition, the fusion protein has a higher molecular weight than its individual component cytokines, and is therefore expected to have a longer serum half-life than IL-24 or the immunostimulatory cytokine alone. This can be particularly beneficial to the immunostimulatory cytokines that have a shorter half-life, such as IL-15. The fusion proteins described herein, with favorable therapeutic efficacy and reduced toxicity, can be used to treat cancer with a greater therapeutic window.

Accordingly, in one aspect, the present disclosure provides a fusion protein comprising an IL-24 polypeptide and an immunostimulatory cytokine.

In some embodiments the immunostimulatory cytokine comprises an IL-15 polypeptide. In some embodiments, the IL-15 polypeptide comprises an amino acid sequence at least 85%, at least 90%, at least 95%, or at least 99% identical to the amino acid sequence of SEQ ID NO: 14. In some embodiments, the IL-15 polypeptide comprises the amino acid sequence of SEQ ID NO: 14. In some embodiments, the C-terminus of the IL-24 polypeptide is fused to the N-terminus of the IL-15 polypeptide. In some embodiments, the IL-24 polypeptide is fused to the IL-15 polypeptide via a linker, such as a linker comprising the amino acid sequence of SEQ ID NO: 33. In some embodiments, the amino acid sequence of the fusion protein comprises the amino acid sequence of SEQ ID NO: 41.

In some embodiments, the immunostimulatory cytokine comprises an IL-12 polypeptide.

In some embodiments, the immunostimulatory cytokine comprises an IL-2 polypeptide.

In some embodiments, the immunostimulatory cytokine stimulates an NK cell and/or a T cell.

In some embodiments, the IL-24 polypeptide comprises an amino acid sequence at least 90% identical to SEQ ID NO: 5. In some embodiments, the IL-24 polypeptide comprises a mutation corresponding to K19R relative to SEQ ID NO: 5. In some embodiments, the IL-24 polypeptide comprises the amino acid sequence of SEQ ID NO: 6.

In some embodiments, the IL-24 polypeptide comprises an amino acid sequence at least 90% identical to SEQ ID NO: 3. In some embodiments, the IL-24 polypeptide comprises the amino acid sequence of SEQ ID NO: 4.

In some embodiments, the IL-24 polypeptide does not comprise amino acids 1-51 of SEQ ID NO: 1.

In another aspect, the present disclosure provides nucleic acids encoding a fusion protein disclosed herein, for example, of the foregoing aspect or any one of the foregoing embodiments.

In some embodiments, the nucleic acid further encodes an N-terminal signal peptide, such as an Insulin signal peptide. In some embodiments, the Insulin signal peptide is a human Insulin signal peptide comprising an amino acid sequence at least 90% identical to the amino acid sequence of SEQ ID NO: 8. In some embodiments, the Insulin signal peptide comprises the amino acid sequence of SEQ ID NO: 8.

In some embodiments, the nucleic acid comprises a nucleotide sequence at least 90% identical to SEQ ID NO: 12. In some embodiments, the nucleic acid comprises the nucleotide sequence of SEQ ID NO: 12.

In another aspect, the present disclosure provides a vector comprising a nucleic acid disclosed herein, for example, of the foregoing aspect or any one of the foregoing embodiments.

In some embodiments, the vector is a viral vector. In some embodiments, the viral vector is an adenovirus vector. In some embodiments, the adenovirus vector comprises a cancer-selective promoter operably linked to E1A and/or E1B genes. In some embodiments, the cancer-selective promoter comprises a PEG-3 promoter or a pGAPE promoter. In some embodiments, the adenovirus vector comprises a chimeric serotype 5/3 adenovirus fiber protein that comprises tail and shaft regions of an Ad.5 fiber protein and a knob region of an Ad.3 fiber protein.

In another aspect, the present disclosure provides a cell comprising a nucleic acid or vector disclosed herein, for example, of the foregoing aspect or any one of the foregoing embodiments. In some embodiments, the cell is an NK cell. In some embodiments, the cell is a T cell.

The present disclosure is also based, in part, upon the ability to maintain NK cell activity and inhibit NK cell exhaustion using IL-24 or its fusion proteins secreted from the NK cells. For example, it is discovered by the present disclosure that NK cells are key immune effector cells mediating the anti-tumor activity of a fusion protein of IL-24 and IL-15. It is therefore contemplated that NK cells engineered to express such a fusion protein are particularly active and sustained, useful as cell therapies for treating cancer. Such NK cell therapies have the additional benefit of not requiring HLA matching between donor and recipient, therefore available as an off-the-shelf therapeutic product.

Accordingly, in another aspect, the present disclosure provides an NK cell comprising a nucleic acid encoding an IL-24 polypeptide operably linked to a heterologous transcription control element.

In some embodiments, the IL-24 polypeptide comprises an amino acid sequence at least 90% identical to SEQ ID NO: 5. In some embodiments, the IL-24 polypeptide comprises a mutation corresponding to K19R relative to SEQ ID NO: 5. In some embodiments, the IL-24 polypeptide comprises the amino acid sequence of SEQ ID NO: 6.

In some embodiments, the IL-24 polypeptide comprises an amino acid sequence at least 90% identical to SEQ ID NO: 3. In some embodiments, the IL-24 polypeptide comprises the amino acid sequence of SEQ ID NO: 4.

In some embodiments, the IL-24 polypeptide does not comprise amino acids 1-51 of SEQ ID NO: 1.

In some embodiments, the nucleic acid further encodes an N-terminal signal peptide, such as an Insulin signal peptide. In some embodiments, the Insulin signal peptide is a human Insulin signal peptide comprising an amino acid sequence at least 90% identical to the amino acid sequence of SEQ ID NO: 8. In some embodiments, the Insulin signal peptide comprises the amino acid sequence of SEQ ID NO: 8.

In some embodiments, the nucleic acid comprises a nucleotide sequence at least 90% identical to SEQ ID NO: 12. In some embodiments, the nucleic acid comprises the nucleotide sequence of SEQ ID NO: 12.

The following embodiments apply to the cell, such as NK cell or T cell, of any one of the foregoing aspects. In some embodiments, the cell secretes the IL-24 polypeptide or the fusion protein. In some embodiments, the cell further comprises an antigen-specific receptor that binds a tumor-associated antigen. In some embodiments, the antigen-specific receptor comprises a chimeric antigen receptor (CAR).

In another aspect, the present disclosure provides pharmaceutical compositions comprising: (a) a fusion protein, nucleic acid, or vector disclosed herein, for example, of any one of the foregoing aspects or embodiments; and (b) a pharmaceutically acceptable carrier or excipient.

In another aspect, the present disclosure provides a method of treating cancer, the method comprising: administering to a subject in need thereof a therapeutically effective amount of the fusion protein, nucleic acid, or vector-containing pharmaceutical composition. The pharmaceutically composition can be administered locally or systemically. In some embodiments, the cancer is a leukemia.

In some embodiments, the cancer is a solid tumor, such as glioblastoma, prostate cancer, melanoma, liver cancer, prostate cancer, kidney cancer, pancreatic cancer, lung cancer, breast cancer, or bone cancer. To treat a solid tumor, the pharmaceutical composition can be administered at a site of the solid tumor. For example, where the pharmaceutical composition comprises the fusion protein, nucleic acid, or vector encapsulated in a microbubble, the method comprises administering the microbubble systemically (e.g., intravenously) and applying focused ultrasound to the site of the solid tumor to disrupt the microbubble. In some embodiments, the method further comprises, prior to administration of the pharmaceutical composition: (a) administering to the subject a penetrating microbubble composition that comprises a microbubble not containing the pharmaceutical composition, for example, systemically (e.g., intravenously); and (b) applying focused ultrasound to the site of the solid tumor to disrupt the microbubble in the penetrating microbubble composition. In some embodiments, the pharmaceutical composition is administered within 4 hours, 2 hours, 1 hour, 30 minutes, 10 minutes, or 5 minutes after administering the penetrating microbubble composition.

In another aspect, the present disclosure provides pharmaceutical compositions comprising: (a) a cell (e.g., NK cell) disclosed herein, for example, of any one of the foregoing aspects or embodiments; and (b) a pharmaceutically acceptable carrier or excipient.

In another aspect, the present disclosure provides methods of treating cancer, the method comprising: administering to a subject in need thereof a therapeutically effective amount of the cell-containing pharmaceutical composition of any one of the foregoing aspects or embodiments. In some embodiments, the pharmaceutical composition is administered by intravenous infusion.

In some embodiments, the method of treatment disclosed herein, for example, of any one of the foregoing aspects or embodiments, further comprises administering to the subject a therapeutically effective amount of an antibody that binds soluble MICA or soluble MICB. Other embodiments and details of the disclosure are presented herein below.

### BRIEF DESCRIPTION OF THE DRAWINGS

FIG. 1, panel A is a schematic illustrating a strategy for molecular cloning of an IL-24S/IL-15 fusion protein, referred to as “FSK” herein, using templates encoding the IL-24S, a linker, and IL-15. FIG. 1, panels B and C are immunoblots showing the expression of the IL-24S/IL-15 fusion protein in transfected HEK-293 cells (panel B) and natural killer (NK)-92 (NK-92; panel C) cells by Western blot using anti-IL-24 and anti-IL-15 antibodies. Abbreviations: PCR, polymerase chain reaction; Ab, antibody.

FIG. 2, panel A is an immunoblot showing the results of prostate cancer DU-145 cells transfected with individual cytokines IL-15 or IL-24S or a IL-24S/IL-15 fusion protein described in FIG. 1, panel A, where the DU-145 cell lysates were immunoblotted for PARP cleavage (“CI PARP;” a molecular marker of cellular apoptosis). FIG. 2, panel B is a graph showing cell viability as measured by a 3-(4,5-dimethylthiazol-2-yl)-2,5-diphenyl tetrazolium bromide (MTT) assay 5 days after treatment. P values were determined by two-way analysis of variance analysis. ***p<0.001; ****p<0.0001.

FIG. 3, panel A is a graph showing the amount of IL-15 present in supernatants of DU-145 prostate cancer cells transfected with IL-24S, IL-15, or IL-24S/IL-15 fusion protein, as determined by ELISA. FIG. 3, panel B is an immunoblot showing the expression of phosphorylated STAT5 (p-Stat5; as a marker for NK-92 cell activation) in NK-92 cells that were stimulated with the supernatant from DU-145 cells transfected with the various constructs. FIG. 3, panel C is a graph showing the number of NK-92 cells after co-culture with supernatant from different cytokine-transfected DU-145 cells. P values were determined by one-way analysis of variance analysis. *P<0.05; ***p<0.001; ****p<0.0001. Abbreviations: ns, not significant; Con, control.

FIG. 4 is a set of graphs showing the results of proliferation assays of glioblastoma (GBM) cell lines IM-PHFA, U87, and U251; and GBM patient-derived cell lines VG2, MDA-GSC-11, and MDA-GSC-23, transduced with adenovirus serotype 5 (Ad.5) vectors encoding IL-15 (Ad.5 IL-15), IL-24S (Ad.5 IL-24S), or an FSK payload (also referred to as Ad.5 FSK and Ad.5 IL-24S/IL-15), or no payload (null; Ad.5 null) at 2500, 5000, 7500, and 10,000 viral particles (VP)/cell. Cell proliferation was measured by MTT assays at 96 hours post-transduction. P values were determined by one-way analysis of variance analysis. *P<0.05; **p<0.01; ***p<0.001. Abbreviations: ns, not significant.

FIG. 5 is a set of graphs and photomicrographs showing the number of GBM cell colonies from cell lines U251 and U87 when cells were transduced with Ad.5 IL-15, Ad.5 IL-24S, Ad.5 IL-24S/IL-15, or Ad.5 null and stained with crystal violet. P values were determined by one-way analysis of variance analysis. ****p<0.0001. Abbreviations: ns, not significant; no, number.

FIG. 6 is a set of immunoblots of U87 and U-251 (also referred to as U251) GBM cells transduced with Ad.5 IL-15, Ad.5 IL-24S, Ad.5 IL-24S/IL-15, or Ad.5 null (5000 vp/cell). At 72 hours post-transduction, the cells were lysed and immunoblotted for PARP cleavage as a molecular marker of cellular apoptosis and GRP78 induction as a molecular marker of ER stress.

FIG. 7, panel A and panel B are a set of graphs showing the concentration (pg/mL) of IL-24 and IL-15, respectively, in the supernatant of GBM GL261 cells transduced with Ad.5 IL-15, Ad.5 IL-24S, Ad.5 IL-24S/IL-15, or Ad.5 null (5000 vp/cell) for 96 hours, as measured by an enzyme-linked immunosorbent assay (ELISA). FIG. 7, panel C is a graph showing the percentage of lysis, as a measure of cytotoxicity, of NK cells (effector cells; “E”) towards GBM GL261 cells (target cells; “T”) at the indicated E/T ratios cultured for 24 hours. The supernatant from the GBM GL261 cells was collected and cytotoxicity was measured using the lactate dehydrogenase (LDH) release assay. P values were determined by one-way analysis of variance analysis. ****p<0.0001. Abbreviation: ns, not significant.

FIG. 8 is a Kaplan-Meier survival curve showing the survival of mice injected with GBM cells to form tumors and transduced one week later with 109 virus particles of Ad.5 IL-15, Ad.5 IL-24S, Ad.5 FSK, or Ad.5 null; or co-transduced with two Ad.5, each encoding IL-24S or IL-15 (Ad.5 IL-24S+Ad.5 IL-15), respectively, via the focused ultrasound (FUS)-double microbubble (DMB; FUS-DMB) method described herein. n=10. The p values from the rank test showed a significant difference (see Table 10).

FIG. 9 is a graph showing the weight of a pancreatic orthotopic tumor from PDX-implanted mice one week after PDX implantation (and tumor growth) and treatment with control NK cells (NKCells) or NK cells genetically engineered to express FSK (NK-92-FSK), IL-15, or IL-24S (NK-92^{IL-24S}).

FIG. 10, panels A-F are a set of graphs showing the percentage of infiltrated CD4⁺ and CD8⁺ T cells (activated CD4 and CD8 cells), CD3⁻NK1.1⁺ NK cells, CD3⁺ NK1.1⁺ NKT cells, CD11c⁺ DCs, and F4/80 immune cells, respectively, analyzed by flow cytometry in tumors on day 14 of mice transduced with Ad.5 IL-15, Ad.5 IL-24S, Ad.5 IL-24S/IL-15, or Ad.5 null with the FUS-DMB method described herehin, after intracranial injection of GBM GL261 cells, as described in FIG. 8. FIG. 10, panel G and panel H depict the relative fluorescence intensity (MFI) (%) of IFNy-expressing CD4 and CD8 T cells. P values were determined by a non-parametric Kruskal-Wallis one-way ANOVA with Dunn's post-test. **p<0.01; ***p<0.001; ****p<0.0001.

FIG. 11, panel A is a schematic showing an experimental design where C57BL/6 mice were injected intracranially with GBM GL261 luciferase (Luc) reporter (GL261-Luc) cells and treated with 108 VP of Ad.5 FSK, Ad.5 IL-24S, or Ad.5 IL-15. 22 days after tumor inoculation, splenocytes were isolated from the mice and co-cultured with irradiated GL261-Luc cells at 37° C. for 2 days. CD4⁺ and CD8⁺ T cells that produced IFNγ or TNFα were analyzed by flow cytometry. FIG. 11, panel B and panel D are a set of graphs showing flow cytometric profiles showing the percentage of TNF&-producing CD4⁺ and CD8⁺ T cells from the experiment described in FIG. 11, panel A. FIG. 11, panel C and panel E are a set of graphs showing the percentage of IFNγ-producing CD4⁺ and CD8⁺ T cells. n=5 mice per group. Statistical significance was determined by using a non-parametric Kruskal-Wallis one-way ANOVA with Dunn's post-test. *P<0.05; **P<0.01, ***P<0.005, ****P<0.001.

FIG. 12 is a set of graphs showing the mRNA levels of IFNg, granzyme-B (GZB), and perforin, in NK-92 cells following treatment with purified IL-15 (50 ng/ml) or IL-24S (1 ug/mL) protein alone or in combination (IL-15+IL-24S) for 24 hours (three left-most panels). To identify the levels of IFNg, granzyme-B (GZB), and perforin, mRNA was isolated and cDNA was prepared and quantified. *P<0.01 vs. controls; **P<0.001 vs. control, N=3 (all panels).

FIG. 13 is a graph showing the relative mRNA level of IL-24 in NK-92 cells were treated with IL-24S at 5 g/mL for 24 hours (h; right panel). Alternatively, NK-92 cells were treated with IL-24S at 5 μg/mL for 12 hours (left panel) and the relative mRNA levels of Granzyme B, Perforin, and CD107a was measured. In each panel, the relative mRNA level is represented as fold change over the NK-92 cell samples not treated with IL-24S. *P<0.05 (right panel).

FIG. 14, panel A and panel B are an immunoblot and a graph, respectively, showing the expression of p-Stat5 (as a marker of NK-92 cell activation) in NK-92 cells when the cells were transfected with control, IL-24S-, IL-15-, or FSK-encoding plasmids.

FIG. 15, panel A is a set of graphs showing the cytotoxic effect of NK-92 cells genetically engineered to express FSK (NK-92-FSK), IL-15, or IL-24S (NK-92^{IL-24S}) on pancreatic ductal adenocarcinoma (PDAC) cells. The genetically engineered NK-92 cells (effector cells) were incubated with 2×10³ PDAC cell lines ASPC1 or KPC at ratios of 1:3, 1:6, or 1:10 PDAC cells: NK cells and the percentage of cell lysis in the respective PDAC cell line was measured. FIG. 15, panel B and panel C are a set of graphs showing the levels of GZB and interferon (INF) g (also referred to as INF-y, INFγ, INF-γ, and INF-g) in the ASPC1 and KPC cells described supra.

FIG. 16 is a set of graphs showing the level of lysis in 10⁴ prostate cancer cell lines DU-145 and PC-3 (target cells) co-cultured with NK-92^{IL-24S} or control NK-92 cells (NK-92ctrl; effector cells) at ratios of 30:1, 10:1, 3:1, and 1:1 in a final volume of 100 μl of RPMI-1640 medium with 5% fetal bovine serum in round-bottom 96-well culture plates at 37° C. for 4 hours. Results depict triplicates and *represents statistical significance between groups in each time point.

FIG. 17, panel A is a schematic showing an experimental design where mice were injected intraperitoneally with anti-CD8, anti-CD4, or anti-NK1.1 antibody on experimental days −3, −1, and 4 relative to GL261-Luc cell injection. Once GL261-Luc tumor-bearing, the mice were then treated with equal virus particles (about 108) of Ad.5 FSK, Ad.5 IL-24S, or Ad.5 IL-15 on day 7 (n=3). FIG. 17, panel B is a graph showing the depletion of CD4-positive, CD8-positive, and NK cell population in blood samples of the mice described supra. Statistical significance was determined by using the student's t test. FIG. 17, panel C is d a graph showing the average size of tumors in the region of interest (ROI) measured from 3 mice per group. Statistical significance was determined by using a non-parametric Kruskal-Wallis one-way ANOVA with Dunn's post-test. *P<0.05; **P<0.01, ***P<0.005, ****P<0.001.

FIG. 18, panel A is a graph showing the transduction efficiency of PSMA CAR-IL-24S in NK-92 cells as measured by flow cytometry when the cells were transduced with a lentiviral vector encoding PSMA CAR-IL-24S. FIG. 18, panel B is a graph showing the proliferation of ^{PSMA}NK-92^{IL-24S} and control NK-92 cells (^{PSMA}NK-92^{Con}). FIG. 18, panel C is a graph showing the percentage of lysis in 2×10³ PSMA over-expressing prostate cancer PC-3 cells (PC-3^{PSMA}; target cells) in a 4 hour LDH release assay when cells were co-cultured with ^{PSMA}NK-92^{IL-24S} or ^{PSMA}NK-92^{Con} (target cells) at ratios of 30:1, 10:1, 3:1, and 1:1. FIG. 18, panel D is a graph showing the relative expression level of NK-92 cell activation markers TIM-3, IFN-G, TIGIT, NKP44, CD69, GZB, and CD107 in the PC-3^{PSMA} cells described supra when co-cultured with ^{PSMA}NK-92^{IL-24S} or ^{PSMA}NK-92^{Con}. To quantify the cell activation markers, total RNA was extracted and analyzed for the indicated marker. * P values were determined by one-way analysis of variance analysis.*p<0.01.

FIG. 19, panel A and panel B are a set of graphs showing the concentration of TNF-a (FIG. 19, panel A, left-most panel), INF-y (FIG. 19, panel A, middle panel), and IL-6 (FIG. 19, panel A, right-most panel), respectively, in humanized mice, as well as the weight of the mice thereof (FIG. 19, panel B), that received 5 injections of IL-24S (5 mg/kg or 10 mg/kg) over the course of 5 days. Mice were euthanized 7 days after the initial injection, and serum samples were analyzed for weight and the indicated cytokines. Values depicted are the average of three mice.

FIG. 20, panel A is a set of graphs showing the percentage of cell lysis in the PDAC cell lines ASPC1 and KPC (target cells) when 10⁴ cells with or without treatment with an antibody that binds soluble MICA (MICA Ab) were co-cultured with control NK cells (NKCells), NK-92 cells genetically engineered to express IL-15, NK-92TL-24S, or NK-92-FSK at a ratio of 3:1 effector: target cells at 37° C. for 24 hours. *p<0.01 vs. non-antibody treated groups. FIG. 20, panel B is a set of graphs showing the percentage of apoptotic ASPC1 and KPC cells, as measured by FACs of Annexin V-stained cells. FIG. 20, panel C is a set of graphs showing the level in pg of INF-g, as measured by ELISA, in the treated ASPC1 and KPC cells described supra.

FIG. 21, panel A and panel B are a set of graphs and photomicrographs, respectively showing the effects of NK-92 cells, ^{PSMA}NK-92^{Con}, or ^{PSMA}NK-92^{IL-24S} on tumor volume in PC-3^{PSMA} xenograft tumors in mice implanted with 106 of the PC-3^{PSMA} cells. FIG. 21, panel C is a graph showing the tumor weight from the same experiment described in FIG. 21, panel A and panel B. FIG. 21, panel D is a graph showing the relative quantity of NK-92 cells in the blood, as measured by flow cytometry, of the mice described supra and treated with ^{PSMA}NK-92^{Con} or ^{PSMA}NK-92^{IL-24S}.

### DETAILED DESCRIPTION

The present disclosure is based, in part, upon the strategy and implementation of a therapeutic fusion protein including IL-24 and an immunostimulatory cytokine (e.g., IL-15, IL-2, or IL-12). Such therapeutic fusion protein retains the cancer-selective cytotoxicity of IL-24 and stimulates proliferation and activation of a wide range of immune cells, such as NK cells and T cells, resulting in enhanced immune responses to tumors. Furthermore, the fusion protein can maintain the low toxicity of IL-24 and certain immunostimulatory cytokines, such as IL-15, to achieve a favorable safety profile. In addition, the fusion protein has a higher molecular weight than its individual component cytokines, and is therefore expected to have a longer serum half-life than IL-24 or the immunostimulatory cytokine alone. This can be particularly beneficial to the immunostimulatory cytokines that have a shorter half-life, such as IL-15. The fusion proteins described herein, with favorable therapeutic efficacy and reduced toxicity, can be used to treat cancer with a greater therapeutic window.

The present disclosure is also based, in part, upon the ability to maintain NK cell activity and inhibit NK cell exhaustion using IL-24 or its fusion proteins secreted from the NK cells. For example, it is discovered by the present disclosure that NK cells are key immune effector cells mediating the anti-tumor activity of a fusion protein of IL-24 and IL-15. It is therefore contemplated that NK cells engineered to express such a fusion protein are particularly active and sustained, useful as cell therapies for treating cancer. Such NK cell therapies have the additional benefit of not requiring HLA matching between donor and recipient, therefore available as an off-the-shelf therapeutic product.

Various components and aspects of the disclosure are described in further detail in the subsections below.

### Definitions

To facilitate an understanding of the present invention, a number of terms and phrases are defined below.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context. It is contemplated that, additionally, there are compositions of the present disclosure that consist essentially of, or consist of, the recited components, and that there are processes and methods according to the present disclosure that consist essentially of, or consist of, the recited steps.

It should be understood that the order of steps or order for performing certain actions is immaterial so long as the present invention remain operable. Moreover, two or more steps or actions may be conducted simultaneously. The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

As used herein, the terms “effective amount” or “therapeutically effective amount” refer to the amount of an active agent (e.g., a fusion protein or vector as provided herein) sufficient to effect beneficial or desired results. An effective amount can be administered in one or more administrations, applications, or dosages and is not intended to be limited to a particular formulation or administration route.

As used herein, the term “operably linked” refers to configurations between sequences within a vector that allow for functional operations to be carried out. For example, when a transcription control element (e.g., promoter or enhancer) is “operably linked” to a protein-coding sequence, the transcription control element is in the correct location and orientation in relation to the protein-coding sequence to control the initiation of transcription by an RNA polymerase.

As used herein, percent “identity” between a query amino acid sequence and a reference amino acid sequence is defined as the percentage of amino acid residues in the query sequence that are identical to the amino acid residues in the reference sequence, after aligning the sequences and introducing gaps, if necessary, to achieve the maximum percent sequence identity. Similarly, percent “identity” between a query nucleic acid sequence and a reference nucleic acid sequence is defined as the percentage of nucleotides in the query sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

As used herein, the term “pharmaceutical composition” refers to the combination of an active agent with a carrier, inert or active, making a composition especially suitable for diagnostic or therapeutic use in vivo or ex vivo.

As used herein, the terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the terms “subject” and “patient” are used interchangeably and refer to an organism to be treated by the methods and compositions provided herein. Such organisms are preferably a mammal (e.g., human, mouse, rat, guinea pig, dog, cat, horse, cow, pig, or non-human primate, such as a monkey, chimpanzee, baboon, and rhesus), and more preferably, a human.

As used herein, the terms “treat”, “treating”, and “treatment” include any effect, for example, lessening, reducing, modulating, ameliorating, inhibiting (such as arresting development, either in a first instance or recurrence), or eliminating, that results in the prevention or improvement of the condition, disease, disorder, and the like, or ameliorating a symptom thereof. Treating can be curing, improving, or at least partially ameliorating (including relieving by regression of a disease state) the disorder. In some embodiments, treating is curing the disease. As used herein, “reducing” or “reduction” of a symptom or symptoms (and grammatical equivalents of this phrase) means decreasing of the severity or frequency of the symptom(s) or elimination of the symptom(s). Treating includes treatment of a disease in a subject, such as a human.

### I. IL-24 Polypeptides

The present disclosure provides various fusion proteins, nucleic acid vectors, and cells related to IL-24 polypeptides. The IL-24 polypeptide incorporated in these disclosures can be a wild-type human IL-24 polypeptide or a functional variant thereof.

In some embodiments, the IL-24 polypeptide is a wild-type human IL-24 polypeptide (e.g., a wild-type human mature IL-24 polypeptide) with amino acid sequences of full-length and mature polypeptides provided in Table 1. It is understood that an N-terminally truncated human IL-24 polypeptide can retain the IL-24 activity. Accordingly, in some embodiments, the IL-24 polypeptide comprises a portion of a wild-type human IL-24 polypeptide, such as the “M4” fragment provided in Table 1. It is understood that a truncated IL-24 polypeptide relative to SEQ ID NO: 3 (e.g., an M4 fragment) is also a mature polypeptide, given the absence of a signal peptide.

TABLE 1
Exemplary IL-24 Polypeptides

| | | SEQ ID |
|---|---|---|
| Description | Amino Acid Sequence | NO |
| Wild-type | MNFQQRLQSLWTLARPFCPPLLATASQMQMVVLPCLGFT | 1 |
| human IL-24 | LLLWSQVSGAQGQEFHFGPCQVKGVVPQKLWEAFWAV | |
| polypeptide | KDTMQAQDNITSARLLQQEVLQNVSDAESCYLVHTLLEF | |
| | YLKTVFKNYHNRTVEVRTLKSFSTLANNFVLIVSQLQPSQ | |
| | ENEMFSIRDSAHRRFLLFRRAFKQLDVEAALTKALGEVDI | |
| | LLTWMQKFYKL | |
| K122R human | MNFQQRLQSLWTLARPFCPPLLATASQMQMVVLPCLGFT | 2 |
| IL-24 | LLLWSQVSGAQGQEFHFGPCQVKGVVPQKLWEAFWAV | |
| polypeptide | KDTMQAQDNITSARLLQQEVLQNVSDAESCYLVHTLLEF | |
| | YLKTVFRNYHNRTVEVRTLKSFSTLANNFVLIVSQLQPSQ | |
| | ENEMFSIRDSAHRRFLLFRRAFKQLDVEAALTKALGEVDI | |
| | LLTWMQKFYKL | |
| Wild-type | QEFHFGPCQVKGVVPQKLWEAFWAVKDTMQAQDNITSA | 3 |
| mature human | RLLQQEVLQNVSDAESCYLVHTLLEFYLKTVFKNYHNRT | |
| IL-24 | VEVRTLKSFSTLANNFVLIVSQLQPSQENEMFSIRDSAHR | |
| polypeptide | RFLLFRRAFKQLDVEAALTKALGEVDILLTWMQKFYKL | |
| K71R mature | QEFHFGPCQVKGVVPQKLWEAFWAVKDTMQAQDNITSA | 4 |
| human IL-24 | RLLQQEVLQNVSDAESCYLVHTLLEFYLKTVFRNYHNRT | |
| polypeptide | VEVRTLKSFSTLANNFVLIVSQLQPSQENEMFSIRDSAHR | |
| | RFLLFRRAFKQLDVEAALTKALGEVDILLTWMQKFYKL | |
| Wild-type | ESCYLVHTLLEFYLKTVFKNYHNRTVEVRTLKSFSTLAN | 5 |
| human IL-24 | NFVLIVSQLQPSQENEMFSIRDSAHRRFLLFRRAFKQLDV | |
| M4 fragment | EAALTKALGEVDILLTWMQKFYKL | |
| K19R human | ESCYLVHTLLEFYLKTVFRNYHNRTVEVRTLKSFSTLAN | 6 |
| IL-24 M4 | NFVLIVSQLQPSQENEMFSIRDSAHRRFLLFRRAFKQLDV | |
| fragment | EAALTKALGEVDILLTWMQKFYKL | |

In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the IL-24 polypeptide comprises the amino acid sequence of SEQ ID NO: 1.

In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 3. In some embodiments, the IL-24 polypeptide comprises the amino acid sequence of SEQ ID NO: 3.

In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 5. In some embodiments, the IL-24 polypeptide comprises the amino acid sequence of SEQ ID NO: 5.

In some embodiments, the IL-24 polypeptide is a variant of wild-type IL-24, comprising one or more amino acid mutations (e.g., substitutions, insertions, and/or deletions) relative to the corresponding wild-type IL-24 amino acid sequence. For example, it is understood that an arginine (R) substitution for lysine (K) at amino acid position 122 corresponding to SEQ ID NO: 1 reduces ubiquitin-mediated degradation of IL-24 in a producer cell, while retaining the activity of IL-24. Corresponding amino acid substitutions can be made in the mature and truncated IL-24, as K71R and K19R, respectively, relative to the amino acid sequence of SEQ ID NO: 3 and SEQ ID NO: 5. Exemplary IL-24 variants are provided in Table 1.

In some embodiments, the IL-24 polypeptide comprises a mutation corresponding to K122R relative to SEQ ID NO: 1. In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 2. In some embodiments, the IL-24 polypeptide comprises the amino acid sequence of SEQ ID NO: 2.

In some embodiments, the IL-24 polypeptide comprises a mutation corresponding to K71R relative to SEQ ID NO: 3. In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 4. In some embodiments, the IL-24 polypeptide comprises the amino acid sequence of SEQ ID NO: 4.

In some embodiments, the IL-24 polypeptide comprises a mutation corresponding to K19R relative to SEQ ID NO: 5. In some embodiments, the IL-24 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 6. In some embodiments, the IL-24 polypeptide comprises the amino acid sequence of SEQ ID NO: 6.

In some embodiments, the IL-24 polypeptide does not comprise amino acids 1-51 of SEQ ID NO: 1.

### Signal Peptide

In some embodiments, the IL-24 polypeptide or a fusion protein comprising IL-24 comprises an N-terminal signal peptide to facilitate protein secretion. Exemplary amino acid sequences of N-terminal signal peptides are provided in Table 2. It is understood that a signal peptide in Table 2 can be fused to the N-terminus of a mature IL-24 sequence (e.g., one provided in Table 1), thereby to generate an IL-24 polypeptide that can be produced and secreted by a producer cell. It is understood that signal peptide sequences are generally cleaved off in the process of protein secretion. As a result, mature IL-24 polypeptides generally do not include signal peptides.

TABLE 2
Exemplary Signal Peptides

| | | SEQ ID |
|---|---|---|
| Description | Amino Acid Sequence | NO |
| Human IL-24 | MNFQQRLQSLWTLARPFCPPLLATASQMQMVVLPCLGFT | 7 |
| signal peptide | LLLWSQVSGAQG | |
| Human Insulin | MALWMRLLPLLALLALWGPDPAAA | 8 |
| signal peptide | | |
| Human Flt3 | MTVLAPAWSPTTYLLLLLLLSGS | 9 |
| signal peptide | | |
| Human BM40 | MRAWIFFLLCLAGRALA | 10 |
| signal peptide | | |
| Human IL-2 | MYRMQLLSCIALSLALVTNS | 11 |
| signal peptide | | |
| Human IL-12B | MCHQQLVISWFSLVFLASPLVA | 50 |
| (p40) sequence | | |
| signal peptide | | |
| Human IL-15 | MVLGTIDLCSCFSAGLPKTEA | 51 |
| signal peptide | | |

In some embodiments, the signal peptide comprises an amino acid sequence at least 80%, at least 85%, at least 90%, at least 91%, at least 92%, at least 93%, at least 94%, at least 95%, at least 96%, at least 97%, at least 98%, or at least 99% identical to a signal peptide sequence in Table 2. In some embodiments, the signal peptide comprises an amino acid sequence in Table 2.

In some embodiments, the signal peptide is an Insulin signal peptide. In some embodiments, the Insulin signal peptide is a human Insulin signal peptide comprising an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the Insulin signal peptide is a human Insulin signal peptide comprising the amino acid sequence of SEQ ID NO: 8. In some embodiments, the Insulin signal peptide has 1, 2, 3, 4, or 5 amino acid substitutions relative to SEQ ID NO: 8.

In some embodiments, the present disclosure provides a nucleic acid encoding an IL-24 polypeptide that incorporates an N-terminal signal peptide (e.g., an Insulin signal peptide). In some embodiments, the nucleic acid comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 12. In some embodiments, the nucleic acid comprises the nucleotide sequence of SEQ ID NO: 12, provided below:

(SEQ ID NO: 12)
ATG GCG CTG TGG ATG CGC CTG CTG CCG CTG CTG GCG
CTG CTG GCG CTG TGG GGC CCA GAT CCG GCG GCG GCG
CAT CAC CAT CAC CAT CAC GAG AAC CTG TAC TTC CAG
GGC ATG CAA GAA TTC CAC TTT GGG CCC TGC CAA GTG
AAG GGG GTT GTT CCC CAG AAA CTG TGG GAA GCC TTC
TGG GCT GTG AAA GAC ACT ATG CAA GCT CAG GAT AAC
ATC ACG AGT GCC CGG CTG CTG CAG CAG GAG GTT CTG
CAG AAC GTC TCG GAT GCT GAG AGC TGT TAC CTT GTC
CAC ACC CTG CTG GAG TTC TAC TTG AAA ACT GTT TTC
AGA AAC TAC CAC AAT AGA ACA GTT GAA GTC AGG ACT
CTG AAG TCA TTC TCT ACT CTG GCC AAC AAC TTT GTT
CTC ATC GTG TCA CAA CTG CAA CCC AGT CAA GAA AAT
GAG ATG TTT TCC ATC AGA GAC AGT GCA CAC AGG CGG
TTC CTG CTA TTC CGG AGA GCA TTC AAA CAG TTG GAC
GTA GAA GCA GCT CTG ACC AAA GCC CTT GGG GAA GTG
GAC ATT CTT CTG ACC TGG ATG CAG AAA TTC TAC AAG
CTC TAG .

In some embodiments, the signal peptide (e.g., Insulin signal peptide) is joined to a mature IL-24 polypeptide by a peptide linker of 1, 2, 3, 4, 5, 6, 7, 8, 9, 10, or more amino acids. In some embodiments, the peptide linker is about 1-10, 2-8, 3-7, or 4-6 amino acids in length. In some embodiments, the signal peptide (e.g., Insulin signal peptide) is joined to the N-terminus of a mature IL-24 polypeptide by a peptide linker within 1, 2, 3, 4, 5, or more amino acids. In other embodiments, the signal peptide (e.g., Insulin signal peptide) is adjacently joined to the N-terminus of a mature IL-24 polypeptide without a peptide linker.

It is understood that the inclusion of an Insulin signal peptide can increase mature protein secretion. Comparison can be made between a first population of producer cells and a second population of producer cells, where the first population of producer cells comprise a nucleic acid encoding a full-length IL-24 polypeptide that incorporates an Insulin signal peptide, and the second population of producer cells comprise a nucleic acid encoding a corresponding full-length IL-24 that incorporates a native (i.e., wild-type IL-24) signal peptide. In some embodiments, secretion of mature IL-24 polypeptide secreted from the first population of producer cells is increased by at least 10%, 20%, 30%, 40%, 50%, 75%, 2-fold, 2.5-fold, 3-fold, 4-fold, or 5-fold, relative to mature IL-24 polypeptide secreted from the second population of producer cells. In some embodiments, the percent or fold increase is in the range of 10% increase to 10-fold increase, 20% increase to 5-fold increase, or 50% increase to 2-fold increase. The amounts of secreted proteins can be measured by any suitable method, such as enzyme-linked immunosorbent assay (see, e.g., International Publication No. WO2019147623).

### II. Immunostimulatory Fusion Proteins

In one aspect, the present disclosure provides a fusion protein comprising IL-24 and an immunostimulatory cytokine. Various methods can be used to decide whether a cytokine is an immunostimulatory cytokine. For example, the cytokine can be incubated with CD8⁺ T cells in a cell culture, optionally in the presence of antigen-presenting cells expressing a class I Major Histocompatibility Complex (MHC) presenting a T cell epitope recognized by the CD8⁺ T cells. Survival and proliferation of the T cells can be assessed by any appropriate technique known in the art, e.g., cell counting, CFSE staining (see Quah and Parish, J. Vis. Exp. (2010) 44:2259), dye exclusion assays of cell viability (e.g. with trypan blue or propidium iodide), Ki-67 staining, tritiated thymidine incorporation, BrdU incorporation (see Gratzner, Science (1982) 218 (4571): 474-75), or by quantitation of ATP levels (e.g. with CellTiter-Glo Cell Viability assay). Activation of T cells can be assessed by cell surface markers such as CD69, CD25, CD44, and CD107a; production of cytokines such as IFNY; and cytotoxicity against target cells (see, e.g., Example 1 below). Development into memory T cells can be assessed by cell surface markers such as CD95, Scal, CD62L, CD27, CD127, and CD44 (see Samji et al., Immunol. Lett. (2017) 185:32-39). T cell exhaustion can be assessed by cell surface markers such as PD-1, LAG3, TIM3, and CTLA4, and by activation of certain transcription factors such as Eomesodermin (see Wherry and Kurachi, Nat. Rev. Immunol. (2015) 15:486-99). In another example, a cytokine can be administered to a subject and its impact on survival, proliferation, activation, memory formation, and/or exhaustion of CD8⁺ T cells can be assessed in vivo. Exemplary immunostimulatory cytokines include but are not limited to IL-15, IL-12, and IL-2.

### IL-15: IL-24 Fusion Proteins

The present disclosure provides various fusion proteins comprising an IL-24 polypeptide and the immunostimulatory cytokine IL-15. Two isoforms of IL-15 mRNA are generated by alternative splicing, and, although both isoforms produce the same mature protein, they differ in their cellular trafficking. The IL-15 polypeptides fused with IL-24 in the present disclosure can be a wild-type human IL-15 polypeptide of either isoform, the mature protein, or a functional variant thereof.

In some embodiments, the IL-15 polypeptide is a wild-type human IL-15 polypeptide with an amino acid sequence of a full-length or mature polypeptide provided in Table 3.

TABLE 3
Exemplary IL-15 Proteins

| | | SEQ ID |
|---|---|---|
| Description | Amino Acid Sequence* | NO |
| Wild-type | MRISKPHLRSISIQCYLCLLLNSHELTEAGIHVFILGCFSAGLP | 13 |
| human IL-15 | KTEANWVNVISDLKKIEDLIQSMHIDATLYTESDVHPSCK | |
| isoform 1 | VTAMKCFLLELQVISLESGDASIHDTVENLIILANNSLSSN | |
| polypeptide | GNVTESGCKECEELEEKNIKEFLQSFVHIVQMFINTS | |
| (GeneBank | | |
| Accession No. | | |
| NP_000576) | | |
| Mature wild- | NWVNVISDLKKIEDLIQSMHIDATLYTESDVHPSCKVTA | 14 |
| type human IL- | MKCFLLELQVISLESGDASIHDTVENLIILANNSLSSNGN | |
| 15 isoform 1 | VTESGCKECEELEEKNIKEFLQSFVHIVQMFINTS | |
| polypeptide | | |
| Wild-type | MVLGTIDLCSCFSAGLPKTEANWVNVISDLKKIEDLIQSM | 15 |
| human IL-15 | HIDATLYTESDVHPSCKVTAMKCFLLELQVISLESGDASI | |
| isoform 2 | HDTVENLIILANNSLSSNGNVTESGCKECEELEEKNIKEF | |
| polypeptide | LQSFVHIVQMFINTS | |
| (GeneBank | | |
| Accession No. | | |
| NP_751915) | | |
| Mature wild- | NWVNVISDLKKIEDLIQSMHIDATLYTESDVHPSCKVTA | 14 |
| type human IL- | MKCFLLELQVISLESGDASIHDTVENLIILANNSLSSNGN | |
| 15 isoform 2 | VTESGCKECEELEEKNIKEFLQSFVHIVQMFINTS | |
| polypeptide | | |

*Italics and underline = wild-type human IL-15 signal peptide of isoform 1 in SEQ ID NO: 13 and isoform 2 in SEQ ID NO: 15

In some embodiments, the IL-15 polypeptide is a variant of wild-type IL-15, comprising one or more amino acid mutations (e.g., substitutions, insertions, and/or deletions) relative to the corresponding wild-type IL-15 amino acid sequence.

In some embodiments, the IL-15 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 13. In some embodiments, the IL-15 polypeptide comprises the amino acid sequence of SEQ ID NO: 13.

In some embodiments, the IL-15 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 14. In some embodiments, the IL-15 polypeptide comprises the amino acid sequence of SEQ ID NO: 14.

In some embodiments, the IL-15 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 15. In some embodiments, the IL-15 polypeptide comprises the amino acid sequence of SEQ ID NO: 15.

A fusion protein of IL-24 and IL-15 disclosed herein can comprise an IL-24 polypeptide linked to the N-terminus or C-terminus of an IL-15 polypeptide. In some embodiments, the IL-24 polypeptide is linked, directly or indirectly, to the N-terminus of the IL-15 polypeptide. In some embodiments, the IL-15 polypeptide is linked, directly or indirectly, to the N-terminus of the IL-24 polypeptide. In some embodiments, the IL-24 polypeptide and the IL-15 polypeptide are indirectly linked by a linker (e.g., peptide linker), such as one disclosed in the “Linkers” subsection below. In some embodiments, the peptide linker comprises the amino acid sequence of SEQ ID NO: 33 or 36. In some embodiments, the fusion protein comprises a signal peptide at the N-terminus, according to the “Signal Peptide” subsection above. In some embodiments, the signal peptide comprises the amino acid sequence of SEQ ID NO: 8.

### IL-12: IL-24 Fusion Proteins

The present disclosure provides various fusion proteins comprising an IL-24 polypeptide and the immunostimulatory cytokine IL-12. IL-12 includes two subunits called IL-12A (p35) and IL-12B (p40). These two subunits can be linked by a peptide linker to form a single chain IL-12 (see, e.g., U.S. Pat. No. 7,226,998). In some embodiments, the peptide linker comprises the amino acid sequence of SEQ ID NO: 36. In some embodiments, IL-12A is linked, directly or indirectly (e.g., via a peptide linker), to the N-terminus of IL-12B. In other embodiments, IL-12B is linked, directly or indirectly (e.g., via a peptide linker), to the N-terminus of IL-12A. The IL-12 polypeptides incorporated in these disclosures can be a wild-type human IL-12 polypeptide or a functional variant thereof.

In some embodiments, the IL-12 polypeptide is a wild-type human IL-12 polypeptide with an amino acid sequence of one or more mature polypeptides provided in Table 4. In some embodiments, the IL-12 polypeptide comprises an IL-12A (p35) fragment and/or an IL-12B (p40) fragment provided in Table 4.

TABLE 4
Exemplary IL-12 Proteins

| | | SEQ ID |
|---|---|---|
| Description | Amino Acid Sequence* | NO |
| Wild-type | RNLPVATPDPGMFPCLHHSQNLLRAVSNMLQKARQTLEFY | 17 |
| human IL- | PCTSEEIDHEDITKDKTSTVEACLPLELTKNESCLNSRETSFI | |
| 12A (p35) | TNGSCLASRKTSFMMALCLSSIYEDLKMYQVEFKTMNAKL | |
| | LMDPKRQIFLDQNMLAVIDELMQALNFNSETVPQKSSLEEP | |
| | DFYKTKIKLCILLHAFRIRAVTIDRVMSYLNAS | |
| Wild-type | IWELKKDVYVVELDWYPDAPGEMVVLTCDTPEEDGITWT | 18 |
| human IL- | LDQSSEVLGSGKTLTIQVKEFGDAGQYTCHKGGEVLSHSLL | |
| 12B (p40) | LLHKKEDGIWSTDILKDQKEPKNKTFLRCEAKNYSGRFTC | |
| | WWLTTISTDLTFSVKSSRGSSDPQGVTCGAATLSAERVRGD | |
| | NKEYEYSVECQEDSACPAAEESLPIEVMVDAVHKLKYENY | |
| | TSSFFIRDIIKPDPPKNLQLKPLKNSRQVEVSWEYPDTWSTP | |
| | HSYFSLTFCVQVQGKSKREKKDRVFTDKTSATVICRKNASI | |
| | SVRAQDRYYSSSWSEWASVPCS | |
| Single-chain | IWELKKDVYVVELDWYPDAPGEMVVLTCDTPEEDGITWT | 19 |
| IL-12 | LDQSSEVLGSGKTLTIQVKEFGDAGQYTCHKGGEVLSHSLL | |
| (B-A) | LLHKKEDGIWSTDILKDQKEPKNKTFLRCEAKNYSGRFTC | |
| | WWLTTISTDLTFSVKSSRGSSDPQGVTCGAATLSAERVRGD | |
| | NKEYEYSVECQEDSACPAAEESLPIEVMVDAVHKLKYENY | |
| | TSSFFIRDIIKPDPPKNLQLKPLKNSRQVEVSWEYPDTWSTP | |
| | HSYFSLTFCVQVQGKSKREKKDRVFTDKTSATVICRKNASI | |
| | SVRAQDRYYSSSWSEWASVPCSGGGSGGGSGGGSGGGS | |
| | RNLPVATPDPGMFPCLHHSQNLLRAVSNMLQKARQTLEFY | |
| | PCTSEEIDHEDITKDKTSTVEACLPLELTKNESCLNSRETSFI | |
| | TNGSCLASRKTSFMMALCLSSIYEDLKMYQVEFKTMNAKL | |
| | LMDPKRQIFLDQNMLAVIDELMQALNFNSETVPQKSSLEEP | |
| | DFYKTKIKLCILLHAFRIRAVTIDRVMSYLNAS | |
| Single-chain | RNLPVATPDPGMFPCLHHSQNLLRAVSNMLQKARQTLEFY | 20 |
| IL-12 | PCTSEEIDHEDITKDKTSTVEACLPLELTKNESCLNSRETSFI | |
| (A-B) | TNGSCLASRKTSFMMALCLSSIYEDLKMYQVEFKTMNAKL | |
| | LMDPKRQIFLDQNMLAVIDELMQALNFNSETVPQKSSLEEP | |
| | DFYKTKIKLCILLHAFRIRAVTIDRVMSYLNASGGGSGGGS | |
| | GGGSGGGSIWELKKDVYVVELDWYPDAPGEMVVLTCDT | |
| | PEEDGITWTLDQSSEVLGSGKTLTIQVKEFGDAGQYTCHKG | |
| | GEVLSHSLLLLHKKEDGIWSTDILKDQKEPKNKTFLRCEAK | |
| | NYSGRFTCWWLTTISTDLTFSVKSSRGSSDPQGVTCGAATL | |
| | SAERVRGDNKEYEYSVECQEDSACPAAEESLPIEVMVDAV | |
| | HKLKYENYTSSFFIRDIIKPDPPKNLQLKPLKNSRQVEVSWE | |
| | YPDTWSTPHSYFSLTFCVQVQGKSKREKKDRVFTDKTSAT | |
| | VICRKNASISVRAQDRYYSSSWSEWASVPCS | |

* Bold = linker

In some embodiments, the IL-12 polypeptide is a variant of wild-type IL-12 or a subunit thereof, comprising one or more amino acid mutations (e.g., substitutions, insertions, and/or deletions) relative to the corresponding wild-type IL-12 amino acid sequence. For example, in some embodiments, the IL-12 polypeptide comprises a mutation that reduces affinity for an IL-12 receptor. In some embodiments, the IL-12 polypeptide comprises an IL-12B (p40) subunit comprising one or more mutations that reduce affinity for IL-12RB1 (see, e.g., Glassman et al. (2021) Cell 184:983-99).

Alternatively, or additionally, in some embodiments, the IL-12 polypeptide is a variant of a wild-type IL-12, comprising an exogenous linker between the subunits. Peptide linkers having proper length and flexibility can be selected such that the single-chain IL-12 has at least 10%, at least 20%, at least 30%, at least 40%, at least 50%, at least 60%, at least 70%, at least 80%, at least 90%, or at least 100% of the activity of a corresponding wild-type IL-12 heterodimer.

In some embodiments, the IL-12 polypeptide comprises an IL-12A amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 17. In some embodiments, the IL-12 polypeptide comprises the amino acid sequence of SEQ ID NO: 17. In some embodiments, the IL-12 polypeptide comprises an IL-12B amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 18. In some embodiments, the IL-12 polypeptide comprises the amino acid sequence of SEQ ID NO: 18.

In some embodiments, the IL-12 polypeptide comprises both an IL-12A amino acid sequence and an IL-12B amino acid sequence.

In some embodiments, the IL-12 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 19. In some embodiments, the IL-12 polypeptide comprises the amino acid sequence of SEQ ID NO: 19.

In some embodiments, the IL-12 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 20. In some embodiments, the IL-12 polypeptide comprises the amino acid sequence of SEQ ID NO: 20.

A fusion protein of IL-24 and IL-12 disclosed herein can comprise one or more polypeptide chains. For example, in some embodiments, an IL-12A subunit and an IL-12B subunit are present in separate polypeptide chains. In some embodiments, the IL-24 polypeptide is linked, directly or indirectly, to the N-terminus of the IL-12A polypeptide. In some embodiments, the IL-24 polypeptide is linked, directly or indirectly, to the N-terminus of the IL-12B polypeptide. In some embodiments, the IL-12A polypeptide is linked, directly or indirectly, to the N-terminus of the IL-24 polypeptide. In some embodiments, the IL-12B polypeptide is linked, directly or indirectly, to the N-terminus of the IL-24 polypeptide.

In some embodiments, a fusion protein of IL-24 and IL-12 disclosed herein comprises an IL-12A subunit and an IL-12B subunit in a single polypeptide. An IL-24 polypeptide can be linked to the N-terminus or C-terminus of an IL-12A and/or IL-12B subunit. In some embodiments, the IL-24 polypeptide is linked, directly or indirectly, to the N-terminus of an IL-12 single-chain polypeptide (B-A or A-B orientation). In some embodiments, a IL-12 single-chain polypeptide (B-A or A-B orientation) is linked, directly or indirectly, to the N-terminus of the IL-24 polypeptide. In some embodiments, the IL-12A polypeptide is linked to the N-terminus of the IL-24 polypeptide and the IL-12B polypeptide is linked to the C-terminus of the IL-24 polypeptide. In some embodiments, the IL-12B polypeptide is linked to the N-terminus of the IL-24 polypeptide and the IL-12A polypeptide is linked to the C-terminus of the IL-24 polypeptide.

In some embodiments, the IL-24 polypeptide, the IL-12A polypeptide, and the IL-12B polypeptide are indirectly linked by one or more linkers (e.g., peptide linker), such as one disclosed in the “Linkers” subsection below. In some embodiments, the peptide linker comprises the amino acid sequence of SEQ ID NO: 33 or 36. In some embodiments, the fusion protein comprises a signal peptide at the N-terminus, according to the “Signal Peptide” subsection above. In some embodiments, the signal peptide comprises the amino acid sequence of SEQ ID NO: 8.

### IL-2: IL-24 Fusion Proteins

The present disclosure provides various fusion proteins comprising an IL-24 polypeptide and the immunostimulatory cytokine IL-2. The IL-2 polypeptides incorporated in these disclosures can be a wild-type human IL-2 polypeptide or a functional variant thereof. In some embodiments, the IL-2 polypeptide is a wild-type human IL-2 polypeptide with an amino acid sequences of a full-length or mature polypeptide provided in Table 5.

TABLE 5
Exemplary IL-2 Proteins

| | | SEQ ID |
|---|---|---|
| Description | Amino Acid Sequence* | NO |
| Wild-type | MYRMQLLSCIALSLALVTNSAPTSSSTKKTQLQLEHLLLD | 21 |
| human IL-2 | LQMILNGINNYKNPKLTRMLTFKFYMPKKATELKHLQCL | |
| polypeptide | EEELKPLEEVLNLAQSKNFHLRPRDLISNINVIVLELKGSE | |
| (GeneBank | TTFMCEYADETATIVEFLNRWITFCQSIISTLT | |
| Accession No. | | |
| NP_000577) | | |
| Mature wild- | APTSSSTKKTQLQLEHLLLDLQMILNGINNYKNPKLTRML | 22 |
| type human | TFKFYMPKKATELKHLQCLEEELKPLEEVLNLAQSKNFH | |
| IL-2 | LRPRDLISNINVIVLELKGSETTFMCEYADETATIVEFLNR | |
| polypeptide | WITFCQSIISTLT | |

*Italics and underline = wild-type human IL-2 signal peptide

In some embodiments, the IL-2 polypeptide is a variant of wild-type IL-2, comprising one or more amino acid mutations (e.g., substitutions, insertions, and/or deletions) relative to the corresponding wild-type IL-2 amino acid sequence.

In some embodiments, the IL-2 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 21. In some embodiments, the IL-2 polypeptide comprises the amino acid sequence of SEQ ID NO: 21.

In some embodiments, the IL-2 polypeptide comprises an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 22. In some embodiments, the IL-2 polypeptide comprises the amino acid sequence of SEQ ID NO: 22.

A fusion protein of IL-24 and IL-2 disclosed herein can comprise an IL-24 polypeptide linked to the N-terminus or C-terminus of an IL-2 polypeptide. In some embodiments, the IL-24 polypeptide is linked, directly or indirectly, to the N-terminus of the IL-2 polypeptide. In some embodiments, the IL-2 polypeptide is linked, directly or indirectly, to the N-terminus of the IL-24 polypeptide. In some embodiments, the IL-24 polypeptide and the IL-2 polypeptide are indirectly linked by a linker (e.g., peptide linker), such as one disclosed in the “Linkers” subsection below. In some embodiments, the peptide linker comprises the amino acid sequence of SEQ ID NO: 33 or 36. In some embodiments, the fusion protein comprises a signal peptide at the N-terminus, according to the “Signal Peptide” subsection above. In some embodiments, the signal peptide comprises the amino acid sequence of SEQ ID NO: 8.

### Linkers

In one aspect, the present disclosure provides a fusion protein comprising IL-24 and an immunostimulatory cytokine, and, in some embodiments, a linker (e.g., a peptide linker) is positioned between the IL-24 polypeptide and the immunostimulatory cytokine, and/or between multiple submits of the immunostimulatory cytokine. In other embodiments, a fusion protein does not comprise a linker.

A linker (e.g., flexible peptide linker) can serve to link or separate two polypeptides, such as two polypeptide domains of a fusion protein. For example, a linker can serve to provide flexibility and/or additional space between the two polypeptides that flank the linker. A linker can comprise a linear amino acid chain of natural and/or synthetic origin. The linkers described herein are designed to retain the biological activities of the polypeptides that it links, by allowing the polypeptides to fold correctly and to be presented properly. The linker may contain repetitive amino acid sequences or sequences of naturally occurring polypeptides.

In certain embodiments, the peptide linker is a flexible linker. The amino acid composition of the linker can be selected with properties that confer flexibility, do not interfere with the structure and function of the other domains of the proteins of the present disclosure, and resist cleavage from proteases. For example, glycine and serine residues generally provide protease resistance. In other embodiments, the sequence of the linker imparts one or more additional functionalities, such as a protease cleavage site to allow cleavage of a fusion protein containing the linker or a ribosomal skipping site to allow for translation of two distinct polypeptides from a fusion mRNA.

The length of the linker can be short, e.g., 0, 1, 2, 3, 4, 5, 6, 7, 8, 9, 10, 11 or 12 amino acid residues, or long, e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, 20-25, or about 20 amino acids in length.

In certain embodiments, the linker includes a (GS)ₙ (SEQ ID NO: 23), (GGS)ₙ (SEQ ID NO: 24), (GGGS)ₙ (SEQ ID NO: 33), (GGSG)ₙ (SEQ ID NO: 34), (GGSGG)ₙ (SEQ ID NO: 35), and (GGGGS)ₙ (SEQ ID NO: 25) sequence, in which n is 1, 2, 3, 4, 5, 6, 7, 8, 9, 10, 11, 12, 13, 14, 15, 16, 17, 18, 19, or 20. In certain embodiments, the linker includes an amino acid sequence selected from SEQ ID NOs: 26-37, as listed in Table 6.

TABLE 6
Exemplary Peptide Linkers

| Linker | Amino Acid Sequence | SEQ ID NO |
|---|---|---|
| (GS)₁₀ | GSGSGSGSGSGSGSGSGSGS | 26 |
| (GGS)₁₀ | GGSGGSGGSGGSGGSGGSGGSGGSGGSGGS | 27 |
| (GGGS)₁₀ | GGGSGGGSGGGSGGGSGGGSGGGSGGGSGGGSG | 28 |
| | GGSGGGS | |
| (GGSG)₁₀ | GGSGGGSGGGSGGGSGGGSGGGSGGGSGGGSGG | 29 |
| | GSGGGSG | |
| (GGSGG)₁₀ | GGSGGGGSGGGGSGGGGSGGGGSGGGGSGGGGS | 30 |
| | GGGGSGGGGSGGGGSGG | |
| (GGGGS)₁₀ | GGGGSGGGGSGGGGSGGGGSGGGGSGGGGSGGG | 31 |
| | GSGGGGSGGGGSGGGGS | |
| (GGGGS)₄ | GGGGSGGGGSGGGGSGGGGS | 32 |
| (GGGGS)₃ | GGGGSGGGGSGGGGS | 33 |
| (GGGGS)₂₀ | GGGGSGGGGSGGGGSGGGGSGGGGSGGGGSGGG | 34 |
| | GSGGGGSGGGGSGGGGSGGGGSGGGGSGGGGSG | |
| | GGGSGGGGSGGGGSGGGGSGGGGSGGGGSGGGG | |
| | S | |
| (GGSGG)₂₀ | GGSGGGGSGGGGSGGGGSGGGGSGGGGSGGGGS | 35 |
| | GGGGSGGGGSGGGGSGGGGSGGGGSGGGGSGGG | |
| | GSGGGGSGGGGSGGGGSGGGGSGGGGSGGGGSG | |
| | G | |
| (GGGS)₄ | GGGSGGGSGGGSGGGS | 36 |
| GGGS- | GGGSGGGGSGGGGSGGGGS | 37 |
| (GGGGS)₃ | | |

### Exemplary Immunostimulatory Fusion Proteins

In one aspect, the present disclosure provides a fusion protein comprising IL-24 and the immunostimulatory cytokine IL-15 provided in Table 7.

In some embodiments, the IL-24 polypeptide (e.g., K71R mature human IL-24; SEQ ID NO: 4) is linked to the C-terminus of the human Insulin signal peptide (SEQ ID NO: 8). In some embodiments, the IL-15 polypeptide (e.g., wild-type human IL-15 isoform 2 polypeptide (GeneBank Accession No. NP_751915); SEQ ID NO: 15) is linked to the C-terminus of the IL-24 polypeptide via a flexible linker, e.g., GGGGSGGGGSGGGGS (SEQ ID NO: 33).

TABLE 7
Exemplary IL-24: IL-15 Fusion Proteins*

| | | SEQ ID |
|---|---|---|
| Description | Amino Acid Sequence | NO |
| IL- | MALWMRLLPLLALLALWGPDPAAAHHHHHHenlyfqgmQEF | 38 |
| 24S(K71R)- | HFGPCQVKGVVPQKLWEAFWAVKDTMQAQDNITSARLL | |
| IL-15 | QQEVLQNVSDAESCYLVHTLLEFYLKTVFRNYHNRTVEV | |
| Fusion Protein | RTLKSFSTLANNFVLIVSQLQPSQENEMFSIRDSAHRRFLL | |
| (FSK) | FRRAFKQLDVEAALTKALGEVDILLTWMQKFYKLGGGG | |
| | SGGGGSGGGGSmvlgtidlcscfsaglpkteaNWVNVISDLKKIE | |
| | DLIQSMHIDATLYTESDVHPSCKVTAMKCFLLELQVIS | |
| | LESGDASIHDTVENLIILANNSLSSNGNVTESGCKECEE | |
| | LEEKNIKEFLQSFVHIVQMFINTS | |
| IL- | QEFHFGPCQVKGVVPQKLWEAFWAVKDTMQAQDNITSA | 39 |
| 24S(K71R)- | RLLQQEVLQNVSDAESCYLVHTLLEFYLKTVFRNYHNRT | |
| IL-15 | VEVRTLKSFSTLANNFVLIVSQLQPSQENEMFSIRDSAHR | |
| Mature Fusion | RFLLFRRAFKQLDVEAALTKALGEVDILLTWMQKFYKLG | |
| Protein | GGGSGGGGSGGGGSmvlgtidlcscfsaglpkteaNWVNVISDLK | |
| | KIEDLIQSMHIDATLYTESDVHPSCKVTAMKCFLLELQ | |
| | VISLESGDASIHDTVENLIILANNSLSSNGNVTESGCKE | |
| | CEELEEKNIKEFLQSFVHIVQMFINTS | |
| IL24-IL-15 | MNFQQRLQSLWTLARPFCPPLLATASQMQMVVLPCLGFTLL | 40 |
| Fusion Protein | LWSQVSGAQGQEFHFGPCQVKGVVPQKLWEAFWAVKDT | |
| | MQAQDNITSARLLQQEVLQNVSDAESCYLVHTLLEFYLK | |
| | TVFKNYHNRTVEVRTLKSFSTLANNFVLIVSQLQPSQENE | |
| | MFSIRDSAHRRFLLFRRAFKQLDVEAALTKALGEVDILLT | |
| | WMQKFYKLGGGGSGGGGSGGGGSNWVNVISDLKKIED | |
| | LIQSMHIDATLYTESDVHPSCKVTAMKCFLLELQVISL | |
| | ESGDASIHDTVENLIILANNSLSSNGNVTESGCKECEEL | |
| | EEKNIKEFLQSFVHIVQMFINTS | |
| IL24-IL-15 | QEFHFGPCQVKGVVPQKLWEAFWAVKDTMQAQDNITSA | 41 |
| Mature Fusion | RLLQQEVLQNVSDAESCYLVHTLLEFYLKTVFKNYHNRT | |
| Protein | VEVRTLKSFSTLANNFVLIVSQLQPSQENEMFSIRDSAHR | |
| | RFLLFRRAFKQLDVEAALTKALGEVDILLTWMQKFYKLG | |
| | GGGSGGGGSGGGGSNWVNVISDLKKIEDLIQSMHIDAT | |
| | LYTESDVHPSCKVTAMKCFLLELQVISLESGDASIHDT | |
| | VENLIILANNSLSSNGNVTESGCKECEELEEKNIKEFLQ | |
| | SFVHIVQMFINTS | |
| IL- | MALWMRLLPLLALLALWGPDPAAAQEFHFGPCQVKGVVPQ | 42 |
| 24S(K71R)- | KLWEAFWAVKDTMQAQDNITSARLLQQEVLQNVSDAES | |
| IL-15 | CYLVHTLLEFYLKTVFRNYHNRTVEVRTLKSFSTLANNF | |
| Fusion Protein | VLIVSQLQPSQENEMFSIRDSAHRRFLLFRRAFKQLDVEA | |
| | ALTKALGEVDILLTWMQKFYKLGGGGSGGGGSGGGGSN | |
| | WVNVISDLKKIEDLIQSMHIDATLYTESDVHPSCKVTA | |
| | MKCFLLELQVISLESGDASIHDTVENLIILANNSLSSNG | |
| | NVTESGCKECEELEEKNIKEFLQSFVHIVQMFINTS | |
| IL- | QEFHFGPCQVKGVVPQKLWEAFWAVKDTMQAQDNITSA | 43 |
| 24S(K71R)- | RLLQQEVLQNVSDAESCYLVHTLLEFYLKTVFRNYHNRT | |
| IL-15 | VEVRTLKSFSTLANNFVLIVSQLQPSQENEMFSIRDSAHR | |
| Mature Fusion | RFLLFRRAFKQLDVEAALTKALGEVDILLTWMQKFYKLG | |
| Protein | GGGSGGGGSGGGGSNWVNVISDLKKIEDLIQSMHIDAT | |
| | LYTESDVHPSCKVTAMKCFLLELQVISLESGDASIHDT | |
| | VENLIILANNSLSSNGNVTESGCKECEELEEKNIKEFLQ | |
| | SFVHIVQMFINTS | |

*Italics and underline = human Insulin signal peptide in SEQ ID NOs: 38 and 42 or human IL-24 signal peptide in SEQ ID NO: 40;
bold italics = histidine tag;
lowercase = TEV protease recognition/cleavage site;
unmarked = K71R mature human IL-24 in SEQ ID NOs: 38-39 and 42-43 or wild-type mature human IL-24 polypeptide in SEQ ID NOs: 40-41;
underline = linker;
bold lowercase = IL-15 signal peptide;
bold = mature wild-type human IL-15 polypeptide

In another aspect, the present disclosure provides a fusion protein comprising IL-24 and the immunostimulatory cytokine IL-12 provided in Table 8.

In some embodiments, the IL-24 polypeptide (e.g., K71R mature human IL-24; SEQ ID NO: 4) is linked to the C-terminus of the human Insulin signal peptide (SEQ ID NO: 8). In some embodiments, the IL-12 polypeptide is linked to the C-terminus of the IL-24 polypeptide via a flexible linker, e.g., GGGGSGGGGSGGGGS (SEQ ID NO: 33).

TABLE 8
Exemplary IL-24: IL-12 Fusion Proteins*

| | | SEQ ID |
|---|---|---|
| Description | Amino Acid Sequence | NO |
| IL- | MALWMRLLPLLALLALWGPDPAAAHHHHHHenlyfqgmQEF | 44 |
| 24S(K71R)- | HFGPCQVKGVVPQKLWEAFWAVKDTMQAQDNITSARLL | |
| IL-12 | QQEVLQNVSDAESCYLVHTLLEFYLKTVFRNYHNRTVEV | |
| Fusion Protein | RTLKSFSTLANNFVLIVSQLQPSQENEMFSIRDSAHRRFLL | |
| | FRRAFKQLDVEAALTKALGEVDILLTWMQKFYKLGGGG | |
| | SGGGGSGGGGSmchqqlviswfslvflasplvaIWELKKDVYVVE | |
| | LDWYPDAPGEMVVLTCDTPEEDGITWTLDQSSEVLGS | |
| | GKTLTIQVKEFGDAGQYTCHKGGEVLSHSLLLLHKK | |
| | EDGIWSTDILKDQKEPKNKTFLRCEAKNYSGRFTCW | |
| | WLTTISTDLTFSVKSSRGSSDPQGVTCGAATLSAERVR | |
| | GDNKEYEYSVECQEDSACPAAEESLPIEVMVDAVHKL | |
| | KYENYTSSFFIRDIIKPDPPKNLQLKPLKNSRQVEVSW | |
| | EYPDTWSTPHSYFSLTFCVQVQGKSKREKKDRVFTDK | |
| | TSATVICRKNASISVRAQDRYYSSSWSEWASVPCSGGG | |
| | GSGGGGSGGGGSRNLPVATPDPGMFPCLHHSQNLLRA | |
| | VSNMLQKARQTLEFYPCTSEEIDHEDITKDKTSTVEAC | |
| | LPLELTKNESCLNSRETSFITNGSCLASRKTSFMMALC | |
| | LSSIYEDLKMYQVEFKTMNAKLLMDPKRQIFLDQNM | |
| | LAVIDELMQALNENSETVPQKSSLEEPDFYKTKIKLCI | |
| | LLHAFRIRAVTIDRVMSYLNAS | |
| IL- | MALWMRLLPLLALLALWGPDPAAAQEFHFGPCQVKGVVPQ | 45 |
| 24S(K71R)- | KLWEAFWAVKDTMQAQDNITSARLLQQEVLQNVSDAES | |
| IL-12 | CYLVHTLLEFYLKTVFRNYHNRTVEVRTLKSFSTLANNF | |
| Fusion Protein | VLIVSQLQPSQENEMFSIRDSAHRRFLLFRRAFKQLDVEA | |
| | ALTKALGEVDILLTWMQKFYKLGGGGSGGGGSGGGGSm | |
| | chqqlviswfslvflasplvaIWELKKDVYVVELDWYPDAPGEM | |
| | VVLTCDTPEEDGITWTLDQSSEVLGSGKTLTIQVKEFG | |
| | DAGQYTCHKGGEVLSHSLLLLHKKEDGIWSTDILKDQ | |
| | KEPKNKTFLRCEAKNYSGRFTCWWLTTISTDLTFSVK | |
| | SSRGSSDPQGVTCGAATLSAERVRGDNKEYEYSVECQ | |
| | EDSACPAAEESLPIEVMVDAVHKLKYENYTSSFFIRDII | |
| | KPDPPKNLQLKPLKNSRQVEVSWEYPDTWSTPHSYFS | |
| | LTFCVQVQGKSKREKKDRVFTDKTSATVICRKNASISV | |
| | RAQDRYYSSSWSEWASVPCSGGGGSGGGGSGGGGSRN | |
| | LPVATPDPGMFPCLHHSQNLLRAVSNMLQKARQTLEF | |
| | YPCTSEEIDHEDITKDKTSTVEACLPLELTKNESCLNSR | |
| | ETSFITNGSCLASRKTSFMMALCLSSIYEDLKMYQVEF | |
| | KTMNAKLLMDPKRQIFLDQNMLAVIDELMQALNENS | |
| | ETVPQKSSLEEPDFYKTKIKLCILLHAFRIRAVTIDRVM | |
| | SYLNAS | |
| IL- | MALWMRLLPLLALLALWGPDPAAAQEFHFGPCQVKGVVPQ | 46 |
| 24S(K71R)- | KLWEAFWAVKDTMQAQDNITSARLLQQEVLQNVSDAES | |
| IL-12 | CYLVHTLLEFYLKTVFRNYHNRTVEVRTLKSFSTLANNF | |
| Fusion Protein | VLIVSQLQPSQENEMFSIRDSAHRRFLLFRRAFKQLDVEA | |
| | ALTKALGEVDILLTWMQKFYKLGGGGSGGGGSGGGGSI | |
| | WELKKDVYVVELDWYPDAPGEMVVLTCDTPEEDGIT | |
| | WTLDQSSEVLGSGKTLTIQVKEFGDAGQYTCHKGGE | |
| | VLSHSLLLLHKKEDGIWSTDILKDQKEPKNKTFLRCE | |
| | AKNYSGRFTCWWLTTISTDLTFSVKSSRGSSDPQGVTC | |
| | GAATLSAERVRGDNKEYEYSVECQEDSACPAAEESLPI | |
| | EVMVDAVHKLKYENYTSSFFIRDIIKPDPPKNLQLKPL | |
| | KNSRQVEVSWEYPDTWSTPHSYFSLTFCVQVQGKSKR | |
| | EKKDRVFTDKTSATVICRKNASISVRAQDRYYSSSWSE | |
| | WASVPCSGGGGSGGGGSGGGGSRNLPVATPDPGMFPC | |
| | LHHSQNLLRAVSNMLQKARQTLEFYPCTSEEIDHEDIT | |
| | KDKTSTVEACLPLELTKNESCLNSRETSFITNGSCLASR | |
| | KTSFMMALCLSSIYEDLKMYQVEFKTMNAKLLMDPK | |
| | RQIFLDQNMLAVIDELMQALNFNSETVPQKSSLEEPDE | |
| | YKTKIKLCILLHAFRIRAVTIDRVMSYLNAS | |

*Italics and underline = human Insulin signal peptide;
bold italics = histidine tag;
lowercase = TEV protease recognition/cleavage site;
unmarked = K71R mature human IL-24;
underline = linker;
bold lowercase italics = IL-12B (p40) sequence signal peptide;
bold = IL-12B (p40) sequence;
bold underline = IL-12A (p35) sequence

In another aspect, the present disclosure provides a fusion protein comprising IL-24 and the immunostimulatory cytokine IL-2 provided in Table 9.

In some embodiments, the IL-24 polypeptide (e.g., K71R mature human IL-24; SEQ ID NO: 4) is linked to the C-terminus of the human Insulin signal peptide (SEQ ID NO: 8). In some embodiments, the IL-2 polypeptide is linked to the C-terminus of the IL-24 polypeptide via a flexible linker, e.g., GGGGSGGGGSGGGGS (SEQ ID NO: 33).

TABLE 9
Exemplary IL-24: IL-2 Fusion Proteins*

| | | SEQ ID |
|---|---|---|
| Description | Amino Acid Sequence | NO |
| IL-24S-IL-2 | MALWMRLLPLLALLALWGPDPAAAHHHHHHenlyfqgmQEF | 47 |
| Fusion Protein | HFGPCQVKGVVPQKLWEAFWAVKDTMQAQDNITSARLL | |
| (K71R) | QQEVLQNVSDAESCYLVHTLLEFYLKTVFRNYHNRTVEV | |
| | RTLKSFSTLANNFVLIVSQLQPSQENEMFSIRDSAHRRFLL | |
| | FRRAFKQLDVEAALTKALGEVDILLTWMQKFYKLGGGG | |
| | SGGGGSGGGGSmyrmqllscialslalvtnsAPTSSSTKKTQLQLE | |
| | HLLLDLQMILNGINNYKNPKLTRMLTFKFYMPKKAT | |
| | ELKHLQCLEEELKPLEEVLNLAQSKNFHLRPRDLISNI | |
| | NVIVLELKGSETTFMCEYADETATIVEFLNRWITFCQS | |
| | IISTLT | |
| IL-24S-IL-2 | MALWMRLLPLLALLALWGPDPAAAQEFHFGPCQVKGVVPQ | 48 |
| Fusion Protein | KLWEAFWAVKDTMQAQDNITSARLLQQEVLQNVSDAES | |
| (K71R) | CYLVHTLLEFYLKTVFRNYHNRTVEVRTLKSFSTLANNF | |
| | VLIVSQLQPSQENEMFSIRDSAHRRFLLFRRAFKQLDVEA | |
| | ALTKALGEVDILLTWMQKFYKLGGGGSGGGGSGGGGSm | |
| | yrmqllscialslalvtnsAPTSSSTKKTQLQLEHLLLDLQMILN | |
| | GINNYKNPKLTRMLTFKFYMPKKATELKHLQCLEEE | |
| | LKPLEEVLNLAQSKNFHLRPRDLISNINVIVLELKGSET | |
| | TFMCEYADETATIVEFLNRWITFCQSIISTLT | |
| IL-24S-IL-2 | MALWMRLLPLLALLALWGPDPAAAQEFHFGPCQVKGVVPQ | 49 |
| Fusion Protein | KLWEAFWAVKDTMQAQDNITSARLLQQEVLQNVSDAES | |
| (K71R) | CYLVHTLLEFYLKTVFRNYHNRTVEVRTLKSFSTLANNF | |
| | VLIVSQLQPSQENEMFSIRDSAHRRFLLFRRAFKQLDVEA | |
| | ALTKALGEVDILLTWMQKFYKLGGGGSGGGGSGGGGSA | |
| | PTSSSTKKTQLQLEHLLLDLQMILNGINNYKNPKLTR | |
| | MLTFKFYMPKKATELKHLQCLEEELKPLEEVLNLAQ | |
| | SKNFHLRPRDLISNINVIVLELKGSETTFMCEYADETA | |
| | TIVEFLNRWITFCQSIISTLT | |

*Italics and underline = human Insulin signal peptide;
bold italics = histidine tag;
lowercase = TEV protease recognition/cleavage site;
unmarked = K71R mature human IL-24;
underline = linker;
bold lowercase italics = wild-type human IL-2 signal peptide;
bold = mature wild-type human IL-2 polypeptide

### Methods of Producing Fusion Proteins

The fusion proteins disclosed herein can be made, for example, using recombinant DNA technologies well known to a skilled person in the art. For example, where the fusion protein comprises a single polypeptide, a nucleic acid sequence encoding the polypeptide can be cloned into an expression vector for expressing this polypeptide. Where the fusion protein comprises two separate polypeptide chains, a first nucleic acid sequence encoding a first polypeptide can be cloned into a first expression vector and a second nucleic acid sequence encoding a second polypeptide can be cloned into a second expression vector. In some embodiments, the expression vector(s) can be stably transfected together into host cells to produce the fusion proteins. Additionally, fusion proteins disclosed herein can be made through tandem fusion, domain insertion, post-translational conjugation, or any suitable method for production of fusion proteins known in the art. For example, fusion proteins disclosed herein can be made through genetic engineering of a fusion gene. This typically involves removing the stop codon from a cDNA sequence coding for the first polypeptide and appending the cDNA sequence of the second polypeptide in-frame through ligation or overlap extension PCR. That DNA sequence will then be expressed by a cell as a single protein.

Clones can be cultured under conditions suitable for bio-reactor scale-up and maintained expression of the fusion proteins. The fusion proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography. For example, a fusion protein may be purified by affinity protein A capture chromatography, such as with a MabSelect SuRe resin (Cytiva), followed by a cation exchange chromatography polishing step, such as with a POROS XS strong cation exchange resin (Thermo Fisher). If higher purity is required, size exclusion chromatography may be used in addition to or in lieu of cation exchange chromatography to polish the protein further and remove unwanted homodimers of various molecular weights. Such size exclusion chromatography can, for example, be performed with a Superdex 200 prep grade resin (Cytiva).

### III. Vectors Encoding the Fusion Proteins

The present disclosure also provides a nucleic acid or an expression vector encoding a fusion protein disclosed herein. The expression vector can be a viral or nonviral vector, suitable for ex vivo transfection or in vivo delivery to a subject. The vectors can be episomal or integrative, e.g., integrating the reprogramming gene into the target cell genome, through homologous recombination or random integration. Exemplary expression vectors include but are not limited to plasmids and virus derived vectors such as cytomegalovirus vector, adenoviral vector, adeno-associated viral (AAV) vector, and retrovirus (e.g., Moloney Murine Leukemia Virus, HIV-1, Avian leukosis virus, or lentiviral vectors).

In some embodiments, the vector further comprises a promoter operably linked to the nucleic acid molecule encoding the fusion protein. In some embodiments, the promoter can be a ubiquitous promoter or a constitutive promoter. Non-limiting examples of constitutive and ubiquitous promoters include RNA pol I promoter, RNA pol II promoter, RNA pol III promoter, cytomegalovirus (CMV), CAG (chicken beta-actin promoter with CMV enhancer), and PGK (phosphoglycerate kinase 1) promoters. In certain embodiments, the promoter can be an inducible promoter that can control when a fusion protein gene is expressed. Non-limiting examples of inducible promoters include tetracycline-regulated promoters (tet on or tet off), including but not limited to the TRE3GV promoter, and steroid-regulated promoters derived from glucocorticoid or estrogen receptors. Alternatively or in addition, the promoter can be a tissue specific promoter. Tissue specific promoters are known in the art, and any appropriate tissue specific promoter may be used to control the expression of fusion proteins in the desired tissue. In some embodiments, the promoter is specific for cancer cells, for example, cancer-selective promoters include, but are not limited to, PEG-3 and pGAPE promoters.

Expression of fusion proteins from a gene therapy vector can also be transient, that is, temporary expression of the fusion protein gene over a limited time span. Transient expression may be achieved by use of a non-integrative vector, where the vector is lost from the cell or cell population over time, or by use of an inducible promoter in an integrative or non-integrative vector that can be manipulated to stop or reduce expression of the gene after a period of time. Notwithstanding, it is understood that certain non-integrative vectors can maintain expression for an extended period of time in a cell, e.g., a non-proliferative cell.

In some embodiments, the vectors disclosed herein can further comprise markers to identify and/or select transduced cells. Examples of selectable markers include visual markers such as green fluorescent protein (GFP), red fluorescent protein (RFP), or fluorescein; epitope markers such as His, c-myc, GST, Flag, or HA tags; enzymatic/nutritional markers such as DHFR (dihydrofolate reductase); or antibiotic resistance markers such as neomycin, puromycin, blasticidin, or hygromycin.

In some embodiments, the expression vector is a viral vector. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors. The virus may be an RNA virus (having a genome that is composed of RNA) or a DNA virus (having a genome composed of DNA). In some embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomaviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In some embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-I (HIV-1)) and toroviruses.

In some embodiments, the expression vector is an AAV. Non-limiting examples of AAV vectors include pAAV-MCS (Agilent Technologies), pAAVK-EFla-MCS (System Bio Catalog No. AAV502A-1), pAAVK-EFla-MCS1-CMV-MCS2 (System Bio Catalog No. AAV503A-1), pAAV-ZsGreen1 (Clontech Catalog No. 6231), pAAV-MCS2 (Addgene Plasmid No. 46954), AAV-Stuffer (Addgene Plasmid No. 106248), pAAVscCBPIGpluc (Addgene Plasmid No. 35645), AAVS1_Puro_PGK1_3×FLAG_Twin_Strep (Addgene Plasmid No. 68375), pAAV-RAM-d2TTA::TRE-MCS-WPRE-pA (Addgene Plasmid No. 63931), pAAV-UbC (Addgene Plasmid No. 62806), pAAVS1-P-MCS (Addgene Plasmid No. 80488), pAAV-Gateway (Addgene Plasmid No. 32671), pAAV-Puro_siKD (Addgene Plasmid No. 86695), pAAVS1-Nst-MCS (Addgene Plasmid No. 80487), pAAVS1-Nst-CAG-DEST (Addgene Plasmid No. 80489), pAAVS1-P-CAG-DEST (Addgene Plasmid No. 80490), pAAVf-EnhCB-lacZnls (Addgene Plasmid No. 35642), and pAAVS1-shRNA (Addgene Plasmid No. 82697). Any suitable serotype can be used, such as AAV-8, for example. These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or fusion proteins can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

### Cancer Terminator Virus

A viral expression vector encoding a fusion protein disclosed herein is, in some embodiments, a cancer terminator virus (CTV) as disclosed in PCT Publication No. WO2014093270, which is incorporated herein by reference in its entirety. In certain embodiments, a CTV contemplated herein includes a tropism-modified adenoviral vector, with the adenovirus fiber being chimeric, comprising an adenovirus serotype 5 fiber in which the fiber knob is from adenovirus serotype 3 (Ad. 5/3 CTV). In certain embodiment, the chimeric adenovirus fiber comprises an adenovirus serotype 5 fiber shaft and an adenovirus serotype 3 fiber knob. This chimera infects cancer cells in a coxsackie adenovirus receptor (CAR)-independent manner, i.e., a payload encoded by this adenoviral construct is expressed in both high and low CAR cancer cells. Furthermore, such a CTV (e.g., an Ad. 5/3 CTV), in some embodiments, comprises a genome comprising a E1A gene, which encodes E1A proteins that promotes adenovirus replication, operably linked to a cancer-specific promoter. Such a CTV can displays cancer cell-specific replication and, as a result, expression of its payload specifically in cancer cells. In some embodiments, the adenovirus genome further comprises a E1B gene under the control of the cancer-specific promoter.

Cancer-specific promoters, which are more active in cancer cells than normal cells of the same cell type, are useful for selective or preferential expression of genes in cancer cells. An exemplary cancer-specific protein is a progression elevated gene-3 (PEG-3) promoter, as described in WO2014093270. Another example is a pGAPE promoter adapted from a growth arrest and DNA damage inducible gene-34 (GADD34) promoter and modified at two basepairs that have been shown to significantly improve cancer-cell specificity. In particular, a pGAPE promoter is modified by two single-point mutations at basepair-260 (G to A) and basepair+159 (C to T), relative to the GADD34 promoter (see Bhoopathi et al., Cancers (2022) 14 (6): 1497).

In some embodiments, such an Ad. 5/3 CTV can comprise a nucleotide sequence encoding a fusion protein described herein as payload, for example, in the E3 region of the adenovirus genome. The nucleotide sequence, is, in some embodiments, operably linked to a ubiquitous promoter or a constitutive promoter. Non-limiting examples of constitutive and ubiquitous promoters include RNA pol I promoter, RNA pol II promoter, RNA pol III promoter, cytomegalovirus (CMV), CAG (chicken beta-actin promoter with CMV enhancer), and PGK (phosphoglycerate kinase 1) promoters. In some embodiments, the fusion protein is under the control of a CMV promoter. It is understood that the payload can still be selectively or preferentially expressed in cancer cells, where the E1A gene and/or E1B genes of the Ad. 5/3 CTV are operably linked to a cancer-specific promoter. Alternatively, the payload can be operably linked to an inducible promoter, cell type-specific promoter, or cancer-specific promoter for better selectivity.

### IV. Cells Expressing IL-24

The present disclosure also provides a cell, such as an immune cell (e.g., an NK cell or a T cell) genetically engineered to express IL-24 or a protein (e.g., fusion protein) comprising IL-24.

The cell may comprise a nucleic acid or a vector encoding the IL-24 or a protein comprising IL-24 described herein. This disclosure also provides producer cells expressing IL-24 or a protein comprising IL-24. In some embodiments, the producer cells are produced by introducing into a cell the nucleic acid or expression vector described herein. The cells can be introduced to the nucleic acid or expression vector by any process including, but not limited to, transfection, transduction of a viral vector, transposition, or gene editing. Any gene editing technique known in the art may be used including, but not limited to, techniques comprising clustered regularly interspaced short palindromic repeats (CRISPR-Cas) systems, zinc finger nucleases, transcription activator-like effector-based nucleases and meganucleases. A cell comprising a heterologous DNA insert e.g., at a target locus or safe harbor site can be referred to as an a producer cell (e.g., an engineered cell) and any such suitable producer cell, in some embodiments, is used in the Methods of Producing Fusion Proteins described herein (see Section II).

Also provided herein are populations of cells comprising a plurality of the cells or immune cells. In some embodiments, the genome of at least 20%, 30%, 40%, 50%, 60%, 70%, 80%, 90%, 95%, 99% or greater of the cells express or produce the IL-24 or a protein comprising IL-24 described herein.

In some embodiments, the cell is a stem cell, a human cell, a primary cell, an hematopoietic cell, an hematopoietic stem cell, an adaptive immune cell, a primary immune cell, an innate immune cell. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte, and follicle cells, etc.). In some embodiments, the cell or immune cell is any cell that can give rise to a pluripotent immune cell. In some embodiments, the cell or immune cell is an innate lymphoid cell, including, but not limited to, natural killer (NK) cells. In some embodiments, the immune cell is a T cell, including, but not limited to, naïve CD8+ T cells, cytotoxic CD8+ T cells, naïve CD4+ T cells, helper T cells, e.g., T_{H}1, T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, effector memory T cells, NKT cells, γδT cells; etc. In some embodiments, the immune cell is a B cell, including, but not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc. In certain embodiments, the immune cell is a myeloid cell, including, but not limited to, macrophages, dendritic cells, myeloid-derived suppressor cells, etc.

In certain embodiments, the present disclosure provides a cell or a population thereof useful in cell therapies. It is contemplated that the cell or a descendant (e.g., differentiated cell in its lineage) can respond to IL-24, resulting in increased survival, increased proliferation, increased activation, and/or reduced exhaustion of the cell or its descendant. Accordingly, the cell engineered to express IL-24 or a protein comprising IL-24 can have improved survival, proliferation, activation, and/or reduced exhaustion relative to a cell not engineered to express the same protein. In certain embodiments, the present disclosure provides an immune cell or a progeny thereof, such as a stem cell (e.g., hematopoietic stem cell) or a progenitor cell, engineered to express IL-24 or a protein comprising IL-24.

It is understood that IL-24 can activate NK cells and reduce exhaustion of NK cells. Accordingly, in some embodiments, the cells expressing IL-24 or a protein comprising IL-24 described herein are NK cells. NK cells form part of the innate immune system and provide rapid responses to innate signals from virally-infected cells manner. NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation. It is contemplated that an engineered NK cell of the present disclosure can be advantageous over a T cell engineered to express the same protein, given that NK cell recognition of target cells (e.g., cancer cells) does not rely on specific major histocompatibility complex (MHC). As a result, an NK cell can be provided as an off-the-shelf cell therapy without additional genetic engineering, for example, deletion of endogenous T-cell receptor genes and insertion of exogenous chimeric antigen receptor genes.

Notwithstanding, it is contemplated that an NK cell of the present disclosure can be further engineered to express a chimeric antigen receptor (CAR), for example, a CAR that binds a tumor-associated antigen, e.g., prostate-specific membrane antigen (PSMA) or prostate stem cell antigen (PSCA).

In some embodiments, the CAR includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The extracellular domain and transmembrane can be linked directly or indirectly. In some embodiments, the extracellular domain and transmembrane are linked by a spacer.

The transmembrane domain in some embodiments is derived either from a natural or from a synthetic source. Where the source is natural, the domain in some aspects is derived from any membrane-bound or transmembrane protein. Alternatively the transmembrane domain in some embodiments is synthetic. In some aspects, the synthetic transmembrane domain comprises predominantly hydrophobic residues such as leucine and valine.

In some embodiments, the linkage is by linkers, spacers, and/or transmembrane domain(s).

In some embodiments, the CAR further includes a spacer, which may be or include at least a portion of an immunoglobulin constant region or variant or modified version thereof, such as a hinge region.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

In some embodiments, upon ligation of the CAR, the cytoplasmic domain or intracellular signaling domain of the receptor activates at least one of the normal effector functions or responses of the immune cell, e.g., NK or T cells engineered to express the receptor.

The receptor, e.g., the CAR, can include at least one intracellular signaling component or components. In some embodiments, the receptor encompasses one or more, e.g., two or more, costimulatory domains (e.g., costimulatory domains of CD28 and/or 4-1BB) and an activation domain, e.g., primary activation domain (e.g., a CD3ζ signaling domain), in the cytoplasmic portion.

In some embodiments, the receptor includes a signaling domain and/or transmembrane portion of a costimulatory receptor. In some aspects, the same receptor includes both the activating and costimulatory components.

In some embodiments, the CAR or other antigen receptor further includes a marker, such as a cell surface marker, which may be used to confirm transduction or engineering of the cell to express the receptor.

For example, in some embodiments, the CAR includes an antibody or fragment thereof, including single chain antibodies (sdAbs, e.g. containing only the VH region), VH domains, and scFvs. In some embodiments, the CAR extracellular antigen-binding domain comprises an anti-PSMA antibody or antigen binding fragment thereof.

In some embodiments, the CAR comprises an extracellular scFv comprising an antigen-binding fragment of an antibody that binds a tumor-associated antigen, linked through a hinge region to a transmembrane region (e.g., a CD8 transmembrane region), a CD28 signaling domain, and a CD3-zeta signaling domain. Exemplary tumor-associated antigens are described in Zarour, et al. Holland-Frei Cancer Medicine. 6th edition. Hamilton (ON): BC Decker (20) and International Patent Application Publication No. WO2020088631.

In other embodiments, the cells expressing IL-24 or a protein comprising IL-24 described herein are T cells or T lymphocytes. T cells or T lymphocytes are a type of lymphocyte that play a central role in cell-mediated immunity and they can be distinguished from other lymphocytes, such as B cells and natural killer cells (NK cells), by the presence of a T-cell receptor (TCR) on the cell surface. There are various types of T cell, as summarized below.

In some embodiments, the cells expressing IL-24 or a protein comprising IL-24 described herein are helper T helper cells (Th cells). Th cells assist other white blood cells in immunologic processes, including maturation of B cells into plasma cells and memory B cells, and activation of cytotoxic T cells and macrophages. Th cells usually express CD4 on their surface. Th cells become activated when they are presented with peptide antigens by MHC class II molecules on the surface of antigen presenting cells (APCs). These cells can differentiate into one of several subtypes, including Th1, Th2, Th17, Th9, or Tfh, which secrete different cytokines to facilitate different types of immune responses.

In some embodiments, the cells expressing IL-24 or a protein comprising IL-24 described herein are cytolytic T cells (Tc cells, or CTLs). CTLs destroy virally-infected cells and tumor cells and are also implicated in transplant rejection. CTLs usually express CD8 on their surface. These cells recognize their targets by binding to antigen associated with MHC class I, which is present on the surface of all healthy nucleated cells.

In some embodiments, the cells expressing IL-24 or a protein comprising IL-24 described herein are memory T cells. Memory T cells are a subset of antigen-specific T cells that persist long-term after an infection has resolved. They quickly expand to large numbers of effector T cells upon re-exposure to their cognate antigen, thus providing the immune system with ‘memory’ against past infections. Memory T cells comprise at least three subtypes: central memory T cells (TCM cells) and two types of effector memory T cells (TEM cells and TEMRA cells). Memory cells may be either CD4+ or CD8+. Memory T cells typically express the cell surface protein CD45RO.

In some embodiments, the cells expressing IL-24 or a protein comprising IL-24 described herein are regulatory T cells (Treg cells). Treg cells, formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus. Two major classes of CD4+ Treg cells have been described: naturally occurring Treg cells and adaptive (or induced) Treg cells. Naturally occurring Treg cells (also known as CD4+CD25+FoxP3+ Treg cells) arise in the thymus and have been linked to interactions between developing T cells with both myeloid (CD1c+) and plasmacytoid (CD123+) dendritic cells that have been activated with TSLP. Treg cells can be distinguished from other T cells by the presence of an intracellular molecule called FoxP3.

Immune cells (e.g., NK cells or T cells) may be derived ex vivo either from a subject's own peripheral blood (autologous), or from peripheral blood of a donor (allogeneic). Alternatively, immune cells may be derived from ex vivo differentiation of stem cells and/or progenitor cells, such as ESCs or HSPC, obtained from the subject (autologous) or a donor (allogeneic). The immune cells may be from a peripheral blood mononuclear cell (PBMC) sample. Immune cells may be activated and/or expanded prior to being transduced with nucleic acid encoding the molecules providing the IL-24 or a protein comprising IL-24. The immune cells of the present disclosure may be made by: (i) isolation of an immune cell-containing sample from a subject or other sources listed above; and (ii) transduction or transfection of the immune cells with one or more nucleic acid sequence(s) encoding IL-24 or a protein comprising IL-24.

For example, in some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, or stem cells. In some embodiments, the NK cells are isolated from pooled CB. The CB may be pooled from 2, 3, 4, 5, 6, 7, 8, 10, or more units. The NK cells may be autologous or allogeneic. NK cells can be detected by specific surface markers, such as CD16, CD56, and CD8 in humans with absence of CD3 expression.

In some embodiments, the starting population of NK cells is obtained by isolating mononuclear cells using ficoll density gradient centrifugation. The cell culture may be depleted of any cells expressing CD3, CD14, and/or CD19 cells and may be characterized to determine the percentage of CD56+/CD3− cells or NK cells.

Alternatively, an immortalized immune cell line which retains its effector function (e.g., a NK cell line or T cell line that retains its lytic function; a plasma cell line that retains its antibody producing function; or a dendritic cell line or macrophage that retains its phagocytic and antigen presentation function) can act as a therapeutic. For example, an immortalized NK cell line called “NK-92” is provided herein (Gong, J. H., Maki, G., & Klingemann, H. G. (1994). Leukemia, 8 (4), 652-658), which is suitable for use as an off-the-shelf immune cell therapy. The NK-92 cell line, named for its establishment in 1992, mirrors the characteristics of NK cells but with broader and greater cytotoxicity. The cell line was established from the blood cells of a patient with lymphoma and has been made available since it was deposited into the American Type Culture Collection in 1998. In all these embodiments, cells expressing IL-24 or a protein comprising IL-24 are generated by introducing a nucleic acid (e.g., DNA or RNA) coding for the protein by one of many means, including transduction with a viral vector, or transfection with DNA or RNA.

Cells can be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

Following expansion, the cells may be stored, such as by cryopreservation. In certain aspects, the cells may be propagated for days, weeks, or months ex vivo as a bulk population.

Expanded NK cells can secrete type I cytokines, such as interferon-y, tumor necrosis factor-a and granulocyte-macrophage colony-stimulating factor (GM-CSF), which activate both innate and adaptive immune cells, as well as other cytokines and chemokines. In some embodiments, the measurement of these cytokines can be used to determine the activation status of NK cells. In addition, other methods known in the art for determination of NK cell activation may be used for characterization of the NK cells of the present disclosure.

The cells (e.g., immune cells) may then be purified, for example, by selecting on the basis of expression of an antigen-binding domain of an antibody. In certain embodiments, the cells are selected by expression of a selectable marker (e.g., a protein, a fluorescent marker, or an epitope tag) or by any method known in the art for selection, isolation and/or purification of the cells. For example, in some embodiments, NK cells are selected by expression of a tag that is operably linked to IL-24 or a protein comprising IL-24 (e.g., a fusion protein) described herein. For example, for detection and/or purification purposes, such a tag that is, in some embodiments, operably linked to IL-24 or a protein comprising IL-24 encompasses a polypeptide or peptide molecule that can serve as a detectable label or targets used in purification e.g., by affinity chromatography (e.g., 6×His, Flag, and V5 tags).

### V. Pharmaceutical Compositions

The present disclosure also provides a pharmaceutical composition comprising IL-24 or a protein (e.g., fusion protein) comprising IL-24.

In some embodiments, the pharmaceutical composition comprising IL-24 or a protein comprising IL-24 comprises a pharmaceutically acceptable carrier, excipient, or stabilizer (see, e.g., Adeboye Adejare, Remington: The Science and Practice of Pharmacy (23d ed. 2020)). Suitable carriers are well known in the art and may include for example, nanoparticles, nanotubes, dendrimers, liposomes, foams, hydrogels, cubosomes, quantum dots, natural drug carriers, exosomes, and macrophages. Acceptable carriers, excipients, or stabilizers are nontoxic to the recipients at the dosages and concentrations, and may comprise phosphate buffered saline solutions, water, emulsions, such as oil/water emulsions, various types of wetting agents, and sterile solutions. In some embodiments, the carrier or excipient comprises buffers such as phosphate, citrate, and other organic acids; antioxidants including ascorbic acid and methionine; preservatives (such as Mercury ((o-carboxyphenyl)thio) ethyl sodium salt (THIO E SAL), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG), TWEEN or PLURONICS.

Pharmaceutical compositions comprising such carriers and/or excipients can be formulated by well-known conventional methods. In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition is formulated as a liquid or the liquid is dried for a solid preparation (for example by spray drying or lyophilization). The lyophilization process is aimed at removal of water and typically involves freezing, primary drying, and secondary drying. To safeguard the peptide molecules from such stresses, typically cryoprotectants are used to improve viability and structural stability. Cryoprotectants include, but are not limited to DMSO, sugars like trehalose and sucrase, polysaccharides, like starch and dextran, or proteins. Solid preparations allow for a long storage time of the pharmaceutical composition. Lyophilized preparations of the pharmaceutical composition can be stored in bulk or in ready-made doses for use such as in a vial. Before administration to a patient, the solid preparation can be dissolved with a suitable solvent such as water.

These pharmaceutical compositions comprising IL-24 or a protein comprising IL-24 can be administered to the subject at a suitable dose. It is preferred that the pharmaceutically acceptable carrier be one which has no detrimental side effects or toxicity under the conditions of use. Preferably, a pharmaceutical composition is sterile and produced according to GMP guidelines.

In some embodiments, the pharmaceutical composition comprising IL-24 or a protein comprising IL-24 is in the form of a lyophilized formulation or an aqueous solution. The pharmaceutical composition can be in dosages suspended in any appropriate pharmaceutical vehicle or carrier in sufficient volume to carry the dosage. In some embodiments, the pharmaceutical composition is administered to a patient by a suitable route, such as, e.g., intravenous administration.

The present disclosure also provides pharmaceutical compositions comprising vectors encoding the proteins, such as a CTV described herein encoding a IL-24 or a protein (e.g., fusion protein) comprising IL-24. For therapeutic use, a vector (e.g., a CTV) preferably is combined with a pharmaceutically acceptable carrier, including buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Adeboye Adejare, Remington: The Science and Practice of Pharmacy (23d ed. 2020). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In some embodiments, a pharmaceutical composition comprising a vector (e.g., a CTV) described herein may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, ethylene vinyl acetate, or poly-D(−)-3-hydroxybutyric acid. Sustained release compositions may also include liposomes that can be prepared by any of several methods known in the art.

Pharmaceutical compositions containing a vector (e.g., a CTV) disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal, and rectal administration. Useful formulations can be prepared by methods known in the pharmaceutical art. For example, see Adeboye Adejare, Remington: The Science and Practice of Pharmacy (23d ed. 2020).

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The present disclosure also provides pharmaceutical compositions comprising one or more microbubble compositions, as described in Sirsi and Borden, Bubble Sci Eng Technol. (2009) 1 (1-2): 3-17 and PCT Publication Nos. WO2014197599, WO2020118271, and WO2022155312, which are incorporated herein by reference in their entirety, comprising the IL-24 or a protein (e.g., fusion protein) comprising IL-24 described herein, or a vector (e.g., CTV) disclosed herein, as payload. Microbubbles are generally 0.5 μm to 10 μm in diameter and filled with a suitable gas (e.g., inert gas). Microbubbles can also be loaded with payloads, such as small molecules, proteins, and vectors for targeted delivery. There are a number of ways to entrap payloads with microbubbles. One technique is to incorporate them into the membrane- or wall-forming materials that stabilize microbubbles. Charged payloads can be stabilized in or onto the surfaces of microbubbles by virtue of electrostatic interactions. Payloads can also be incorporated into the interior of microbubbles (gas-filled microspheres). Another way to entrap drugs in microbubbles is to create a layer of oil (e.g., triacetin) to stabilize the outer surface of the bubble. Hydrophobic paylods can then be incorporated into the oil layer (see, e.g., U.S. Patent Application No. US20100158813, as well as Dijkmans, P. A., et al. European Journal of Echocardiography 5.4 (2004): 245-246; and Mohr, J. P., et al. Elsevier Health Sciences, 2011). The payloads can be released by applying focused ultrasound at desirable locations (see the “Therapeutic Methods” section below).

For example, in some embodiments, described herein are two microbubble compositions, the first microbubble composition comprising first microbubbles and not comprising IL-24 or a protein comprising IL-24 (e.g., empty first microbubble); and a second microbubble composition comprising second microbubbles complexed with a payload (e.g., IL-24 or a protein (e.g., fusion protein) comprising IL-24 described herein, or a vector (e.g., CTV) disclosed herein).

In some embodiments, administering microbubble compositions to a target tissue comprises injection into or adjacent to the target tissue, or topical application to the target tissue, such that the microbubble composition is formulated accordingly as described above. In some embodiments, the composition is delivered distally to the target tissue, but is formulated to traffic the payload to the target tissue. For example, in some embodiments, the microbubble compositions are formulated for intravenous administration, as described above. The microbubbles can be delivered to the target tissue by circulation and release of the payload when focused ultrasound is applied at the target tissue.

Microbubbles can also be coated with a targeting moiety that selectively binds cells at a target tissue to further enrich the microbubbles at the target tissue. The targeting moiety can be directly complexed to an outer surface of the microbubbles, such as by covalent or non-covalent interaction. Complexing can be indirect, such that the microbubble outer surface and the targeting moiety are separated by one or more other molecules joining the two, via covalent or non-covalent interactions. In general, a targeting moiety is a moiety able to bind to or otherwise associate with a biological entity (e.g., a membrane component, a cell surface receptor, cell specific membrane antigen, or the like), with a higher affinity than one or more non-target biological entities (e.g., cell surface components of one or more different tissues). Non-limiting examples of targeting moieties include an antibody, an antibody fragment, a binding protein, a binding protein fragment, a receptor, a receptor fragment, a receptor ligand, a peptide, a polypeptide, a polynucleic acid, a polysaccharide, a lipid, a polymer, or any combination of molecules thereof. The targeting moiety can bind a tumor-associated antigen, a tissue specific antigen, a vascular associated antigen, etc. Illustrative examples of targeting moieties that traffic to bone are described in U.S. Patent Publication Nos. US20120028350A1, US20160052968A1, US20040038946A1, and US20180208650A1, each of which is incorporated herein by reference. A targeting moiety typically allows a payload to become localized at a particular targeting site to a higher degree than elsewhere in the body of the subject, or to a higher degree at the target site than would be accomplished in the absence of the targeting moiety. In some embodiments, the microbubbles compositions described herein are complexed with a targeting moiety that traffics the microbubbles to a particular tissue, such as a cancer tissue, cancer vasculature, or a bone tissue. In some embodiments, the first and second microbubbles both contain a targeting moiety, which may be the same or different. In some embodiments, the first and second microbubbles include a targeting moiety that binds the same target. In some embodiments, the targeting moiety specifically binds a particular protein, a particular cell, or a particular tissue.

### VI. Therapeutic Methods

It is contemplated that the fusion proteins, vectors, immune cells, and pharmaceutical composition disclosed herein can be used, alone or in combination with other therapeutic agents, to treat a disease or condition associated with insufficient immune cell activity, such as cancer. Accordingly, the present disclosure provides a method of treatment (e.g., for treating cancer), comprises administering a fusion protein, vector, immune cell, or pharmaceutical composition disclosed herein to a subject in need thereof.

In some embodiments, the cancer to be treated is a solid tumor, such as brain cancer, for example Glioblastoma (GBM), prostate cancer, melanoma, liver cancer, prostate cancer, kidney cancer, pancreatic cancer, lung cancer, breast cancer, or bone cancer.

Various routes of administration are contemplated herein and depending upon the composition being described it will be understood by one of skill in the art which routes are suitable. For example, non-limiting examples of contemplated routes for administering IL-24 or a protein (e.g., fusion protein) comprising IL-24 described herein include intravenous, intraarterial, intraperitoneal, intramuscular, intratumoral, subcutaneous, intrapleural, intrathecal, intracavitary, perfusion through a catheter, and direct intralesional injection. Non-limiting examples of contemplated routes for administering the vectors encoding fusion proteins described herein include intravenous, intratumoral, local (e.g., transdermal patch), inhalation, ingestion, topical, parenteral, intravenous, peritoneal, intramuscular, intraarterial, and via microbubbles as described herein. Meanwhile, it is contemplated that cells that express IL-24 as described herein can be administered intravenously.

### Delivery of Therapeutic Agents by Microbubble

The present disclosure provides a method of treatment (e.g., for treating cancer), comprising administering one or more microbubble compositions (e.g., by a method described in PCT Publication No. WO2022155312, which is incorporated herein by reference in its entirety) to a subject in need thereof, whereby at least one of the microbubble compositions comprises a fusion protein, vector, or pharmaceutical composition disclosed herein. For example, contemplated herein is a method of treatment comprising administering a microbubble composition that comprises microbubbles comprising a fusion protein, vector, or pharmaceutical composition disclosed herein. The method can further comprise applying a focused ultrasound to a target tissue to disrupt the microbubbles. In certain embodiments, the focused ultrasound is applied within 5 minutes, 10 minutes, 15 minutes, 20 minutes, 25 minutes, or 30 minutes after administration of the microbubble composition.

In certain embodiments, the method comprises (a) administering to a subject a first microbubble composition, the first microbubble composition comprising first microbubbles and not comprising an active agent (e.g., empty microbubbles); (b) applying a first focused ultrasound to a target tissue to disrupt the first microbubbles; (c) administering to the subject a second microbubble composition after the first ultrasound administration, the second microbubble composition comprising second microbubbles loaded with a payload (e.g., active agent), such as the fusion protein, vector, or pharmaceutical composition disclosed herein; and (d) applying a second focused ultrasound to the target tissue to disrupt the second microbubbles and release the active agent to the target tissue. In certain embodiments, the first focused ultrasound is applied within 5 minutes, 10 minutes, 15 minutes, 20 minutes, 25 minutes, or 30 minutes after administration of the first microbubble composition. In certain embodiments, the second focused ultrasound is applied within 5 minutes, 10 minutes, 15 minutes, 20 minutes, 25 minutes, or 30 minutes after administration of the second microbubble composition. The first microbubble composition, also referred to herein as a penetrating microbubble composition, can increase penetration of the second microbubble composition that comprises a payload. In certain embodiments, the second microbubble composition is administered from about 1 second to about 4 hours, about 5 seconds to about 3 hours, about 15 seconds to about 2 hours, about 20 seconds to about 1 hour, or about 60 seconds to about 30 minutes after the administration of the first microbubble composition. In certain embodiments, the second focused ultrasound is applied from about 1 second to about 4 hours, about 5 seconds to about 3 hours, about 15 seconds to about 2 hours, about 20 seconds to about 1 hour, or about 60 seconds to about 30 minutes after the first focused ultrasound is applied.

In certain embodiments, the first and/or second microbubbles have a mean or median diameter of about 1 micron to about 50 microns, about 1 micron to about 25 microns, about 1 micron to about 10 microns, or about 1 micron to about 5 microns. In some embodiments, the first microbubbles have a mean or median diameter of about 1, 2, 3, 4, or 5 microns.

In certain embodiments, the first and/or second microbubbles comprise a targeting moiety. In some embodiments, the first microbubbles contain a targeting moiety. In some embodiments, the second microbubbles contain a targeting moiety. In some embodiments, the first and second microbubbles both contain a targeting moiety, which may be the same or different. In embodiments, the first and second microbubbles include a targeting moiety that binds the same target. In embodiments, the targeting moiety specifically binds a particular protein, a particular cell, or a particular tissue. In some embodiments, the targeting moiety binds a tumor-associated antigen, e.g., selected from HER2, CEA, PSA, MUC1, PSMA, CA19-9, EpCAM, GPC3, mesothelin (MSLN), and EGFR. In some embodiments, the targeting moiety binds a tissue specific antigen, e.g., selected from Glycoprotein 2, Cadherin-9, GFAP, nestin, Tuj-1, Thymocyte antigen 1 (Thy1)/CD90, Desmin, Cx43. In some embodiments, the targeting moiety comprises a VEGF polypeptide or single-chain variant thereof, which binds a VEGF receptor. Non-limiting examples of VEGF polypeptides and single-chain variants thereof useful as targeting moieties are provided in US20080312410A1, which is incorporated herein by reference. In some embodiments, the targeting moiety comprises a VCAM1 antibody or epitope-binding fragment thereof. In some embodiments, the targeting moiety comprises a PSMA antibody or epitope-binding fragment thereof.

In some embodiments, the first microbubble composition is administered in an amount effective to increase delivery of the active agent to the target tissue, such as after first administering the first microbubble composition and first ultrasound administration at the target tissue. In some embodiments, the first microbubble composition is administered in an amount effective to increase delivery of the active agent across the blood-brain barrier, such as after a first administering the first microbubble composition and first ultrasound administration at the target tissue. In some embodiments, the increase in delivery is with respect to administration of the second microbubble composition and second ultrasound administration in the absence of the first microbubble composition and first ultrasound administration. In some embodiments, administration of the first microbubble composition and first ultrasound administration increases delivery of the active agent delivered by the second microbubble composition and second ultrasound administration by about or more than about 25%, 50%, 100%, 200%, 300%, or more.

### Combination Therapies

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. In some embodiments, delivery is such that the reduction of a symptom, or other parameter related to the disorder is greater than what would be observed with one treatment delivered in the absence of the other. The effect of the two treatments can be partially additive, wholly additive, or greater than additive.

Contemplated herein are combination therapies including administration of cells (e.g., NK cells) described herein expressing IL-24 as well as one or more antibodies that bind soluble MICA or MICB. It is understood that soluble (e.g., shedded) MICA and MICB can neutralize NKG2D, an activating receptor on NK cells. Accordingly, antibodies that selectively bind soluble MICA and/or MICB, but not transmembrane MICA and/or MICB, may increase the activity of an NK cell therapy disclosed herein. Exemplary anti-soluble MICA antibodies are described in U.S. Patent Publication No. 20200299380, incorporated herein by reference. In certain embodiments, the present disclosure provides a method of treating cancer, the method comprising administering an NK cell therapy disclosed herein and an antibody that binds soluble MICA or MICB.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Construction of an IL-24S/IL-15 Fusion Protein

This example describes the design and construction of a IL-24S/IL-15 fusion protein, also referred to herein as “FSK,” and an expression plasmid expressing the same.

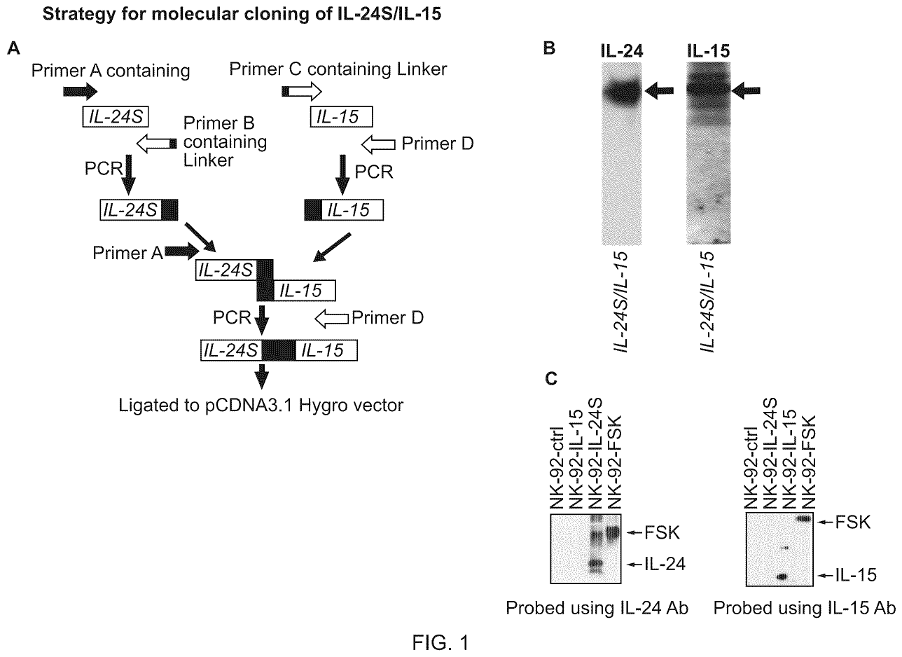

Briefly, a polynucleotide encoding an Insulin signal peptide (SEQ ID NO: 8) and a mature human IL-24 with a K122R mutation (SEQ ID NO: 4), was linked to a polynucleotide encoding a mature human IL-15 (SEQ ID NO: 14) to encode a fusion protein called IL-24S/IL-15 (SEQ ID NO: 38; also referred to herein as “FSK”). As used herein in the Examples section, “IL-24 superkine” or “IL-24S” refers to a combination of two technical features in a polynucleotide: an Insulin signal peptide (SEQ ID NO: 8) and a K122R mutation in IL-24 corresponding to SEQ ID NO: 4. The strategy for the cloning of the IL-24S/IL-15 fusion construct is schematically presented in FIG. 1, panel A. Briefly, the human IL-24S cDNA was amplified with primers A and B, and the human IL-15 cDNA was amplified with primers C and D. The PCR products of IL-24S and IL-15 were combined and subjected to an additional round of PCR with primers A and D. Because primers B and C contained the linker sequence encoding GGGGSGGGGSGGGGS (SEQ ID NO: 33), the IL-24S and IL-15 genes (IL-24S/IL-15) were connected by a flexible linker after the overlap extension PCR. The IL-24S/IL-15 gene was then cloned into a pCDNA3.1 mammalian expression vector. The vector was then transfected into HEK-293 cells (FIG. 1, panel B) or NK-92 cells (FIG. 1, panel C), the respective cells were lysed, and the lysates were analyzed by SDS-PAGE and Western blotting using antibodies against human IL-24 and IL-15.

### Example 2. In Vitro Activity of IL-24S/IL-15 Fusion Protein

This example describes the determination of IL-24S/IL-15 protein activity in vitro by direct expression in cancer cells and treatment of cancer cells with secreted protein.

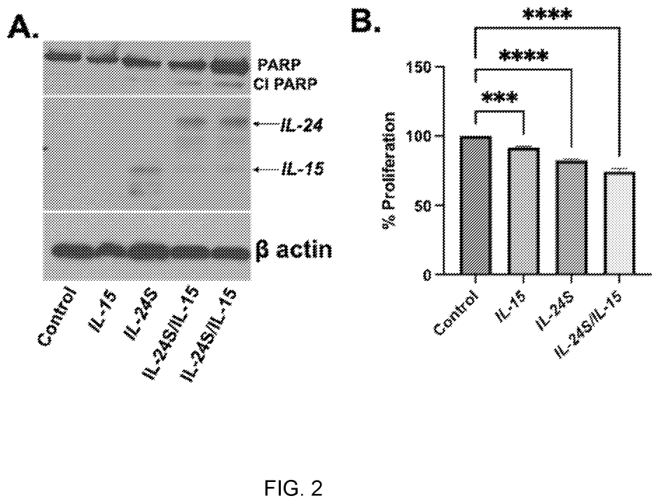

Briefly, DU-145 cells, a human castration-resistant prostate cancer cell line, were transfected with 2.5 μg of IL-24S/IL-15, a single protein construct (IL-24S or IL-15), or the empty vector as a control. 72 hours post-transfection, cells were lysed and immunoblotted for PARP cleavage, as a molecular marker of cellular apoptosis (FIG. 2, panel A). The results showed PARP cleavage in the IL-24S/IL-15- and IL-24S-treated DU-145 cells and confirmed that the IL-24S/IL-15 fusion protein retained biological function of IL-24S in vitro.

To determine biological activity of the secreted IL-24S/IL-15 fusion protein, DU-145 cells were treated with supernatant collected from the transfected cell clones and the anti-proliferative changes were determined by 3-(4,5-Dimethylthiazol-2-yl)-2,5-Diphenyltetrazolium Bromide (MTT) assay. The MTT assay was used to measure cellular metabolic activity as an indicator of cell viability, proliferation, and cytotoxicity. This colorimetric assay is based on the reduction of a yellow tetrazolium salt 3-(4,5-dimethylthiazol-2-yl)-2,5-diphenyltetrazolium bromide to purple formazan crystals by metabolically active cells. Briefly, 1,000 (DU-145) cells were seeded per 96 well in 100 μl medium, cultured overnight, and treated with the different supernatants (control, IL-15, IL-24S, and IL-24S/IL-15) the next day. Cell viability was measured by MTT assay 5 days after treatment. P values were determined by two-way analysis of variance analysis. ***p<0.001; ****p<0.0001. The MTT data showed that supernatant enriched with fusion proteins were able to inhibit the proliferation of cancer cells (FIG. 2, panel B).

### Example 3. Activation of NK-92 and Primary NK Cells by IL-24S/IL-15 Fusion Protein In Vitro

This example describes the determination of IL-24S/IL-15 protein activation of NK-92 cells and primary NK cells in vitro.

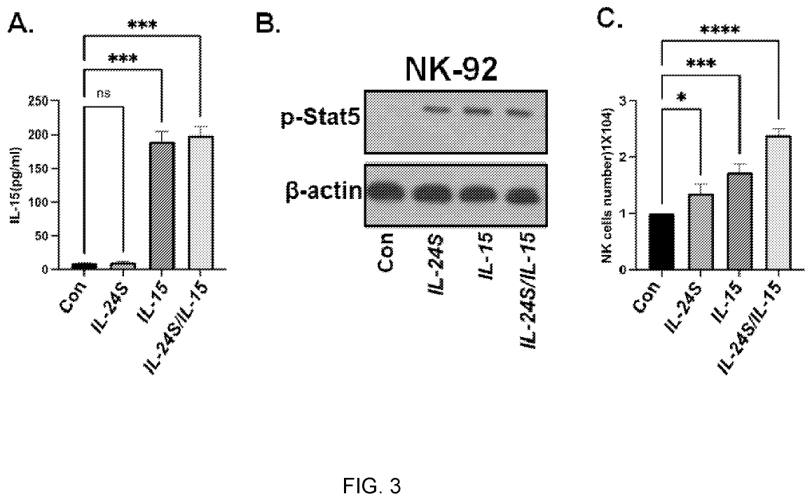

Briefly, DU-145 cells were transfected with 20 μg of different constructs for 72 hours in 10-cm dishes. Supernatants were harvested, and the amounts of IL-15 in the supernatants were determined by ELISA (FIG. 3, panel A). The supernatants were concentrated and added to cultured NK-92 cells for 4 days. NK cell activation was assessed by Western blotting detecting STAT5 phosphorylation (p-Stat5) in NK cells (FIG. 3, panel B). NK-92 cell proliferation over 4 days was measured by carboxyfluorescein succinimidyl ester (CFSE) staining (FIG. 3, panel C). The results showed that the supernatants from the IL-24S/IL-15 fusion-protein-transfected cells induced proliferation of NK-92 cells that was higher than proliferation of NK-92 cells cultured with the supernatant from either of the individual cytokines IL-24S or IL-15.

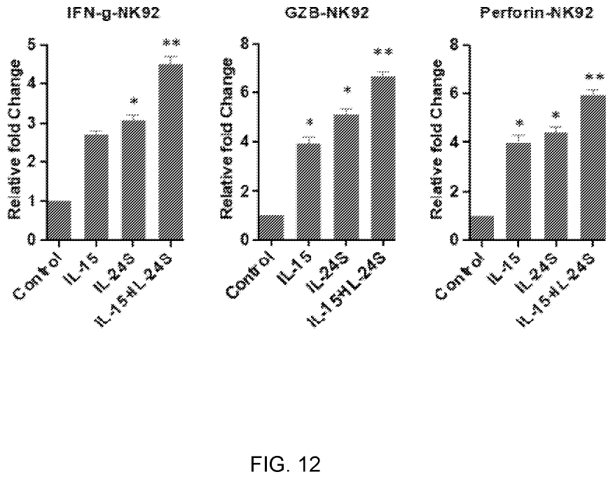

In another experiment, in NK-92 cells, it was confirmed that IL20R1 and IL20R2 receptors were present (data not shown), which was relevant as IL-24 binds to distinct type II cytokine heterodimeric receptor complexes, IL20R1/IL20R2, IL22R1/IL20R1, and IL22R1/IL20R2. Using Western blotting, it was confirmed that the NK-92 cells responded to exogenous, purified IL-24S protein by enhancing the expression of multiple NK cell activation markers, including INFγ, granzyme-B (GZB), and perforin (FIG. 12). It was observed that the expression of INFγ, GZB, and perforin was further enhanced when IL-24S was combined with IL-15 (not fused; FIG. 12).

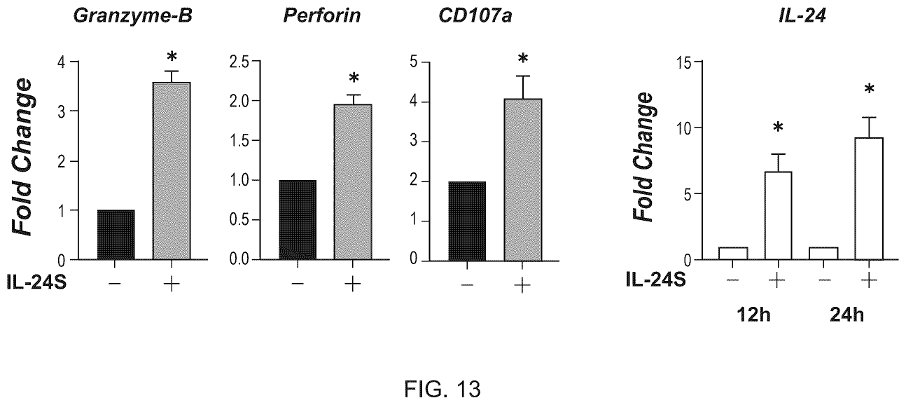

In another experiment, NK-92 cells were treated with IL-24S (5 μg/mL) for 12 hours and the expression of Granzyme B, Perforin, and CD107a mRNAs, markers of NK-92 cell activation, was analyzed using qPCR (FIG. 13, left panel). When the NK-92 cells were treated with IL-24S for 24 hours, endogenous IL-24 mRNA was also induced, likely through an autocrine/paracrine loop (FIG. 13, right panel).

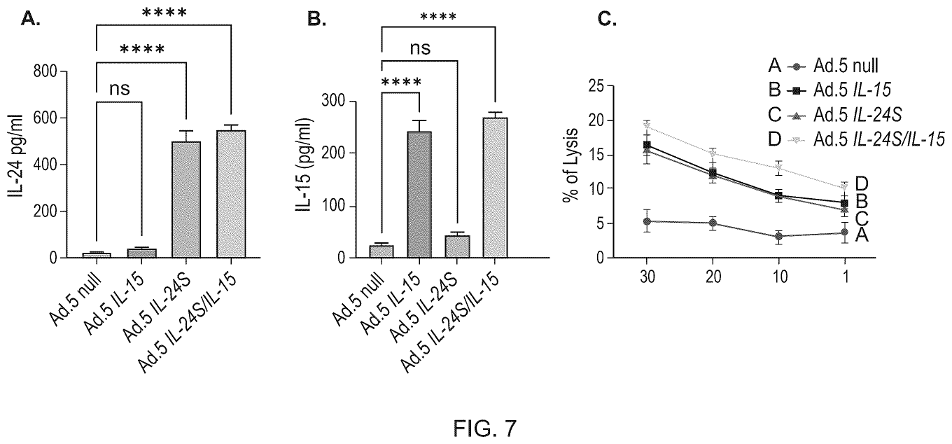

In another experiment, primary NK cells from C57BL/6 mice were enriched and incubated with supernatant collected from adenovirus (Ad) encoding a payload-infected cells. The amounts of IL-24 protein and IL-15 protein secreted to the supernatant were measured by ELISA, as shown in FIG. 7, panel A and panel B, respectively. The cytotoxicity of expanded NK cells toward GL261 (a murine GBM cell line) was measured using the LDH release assay at 30:1 effector-to-target (E:T) ratios for 4 hours (FIG. 7, panel C). The results showed that the fusion protein produced by the NK cells synergistically activated the NK cells more effectively than IL-15 alone or IL-24 alone.

### Example 4. Adenoviruses Engineered to Express IL-24S/IL-15 and Assessment of In Vitro Antitumor Effects

This example illustrates adenoviruses engineered to express IL-24S/IL-15 fusion proteins. The example also describes assessment of in vitro antitumor effects of the adenoviruses.

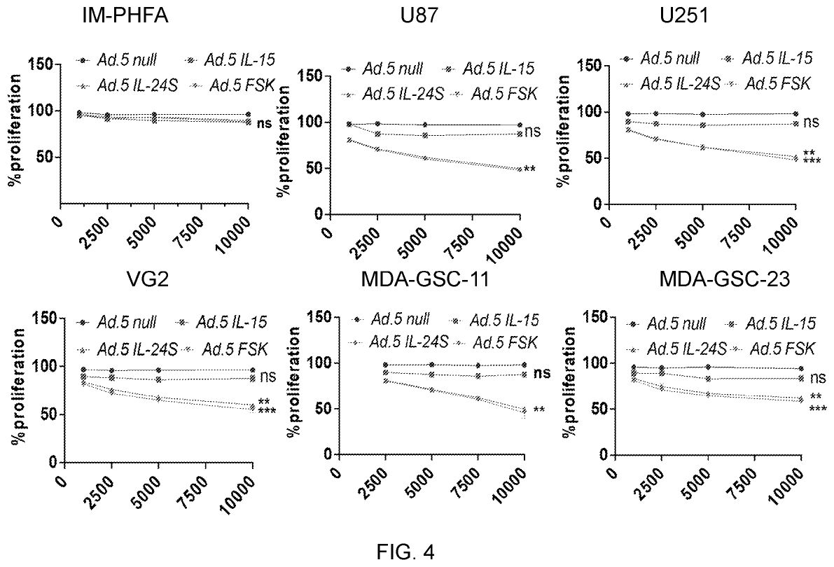

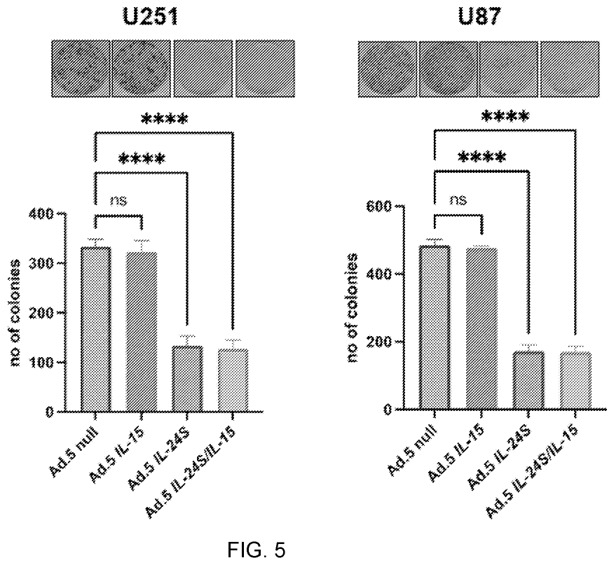

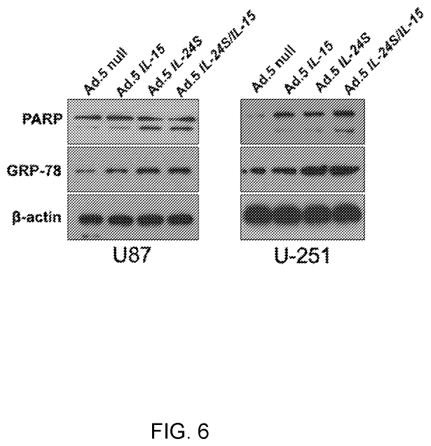

Briefly, multiple additional biological activities expressed by IL-24S were assessed. Adenovirus serotype 5 (Ad.5) encoding a control plasmid or encoding IL-24S (Ad.5-IL-24S), IL-15 (Ad.5 IL-15), or IL-24S/IL-15 (Ad.5-IL-24S/IL-15) were generated following standard protocols. First, it was evaluated whether these Adenoviruses (Ads) displayed any cytotoxic effects in immortal primary human fetal astrocytes (PHFA) cells. As shown in FIG. 4, the viruses did not induce growth suppression in PHFA cells, even when administered at high multiplicity of infection (MOI). Next, a series of established GBM cells (U87 and U251) and GBM patient-derived cell lines (VG2, GSC-23 (MDA-GSC-23), and GSC-11 (MDA-GSC-23)) were tested and found to respond similarly to Ad.5-IL-24S/IL-15 and Ad.5-IL-24S. Ad.5-IL-15 had a minimal non-significant effect on growth, suggesting the IL-24S/IL-15 fusion protein mediated growth arrest was mediated by IL-24S, not IL-15 (FIG. 4). The results showed that, similar to IL-24S, the IL-24S/IL-15 fusion protein-mediated effects were cancer-specific, as this molecule did not produce any growth suppressive or toxic effects on PHFA cells. Colony formation assays were used to determine long-term effects of treatment on proliferation of the fusion protein in two cell lines U87 and U-251 (2500 viral particles (vp)/cell; FIGS. 5). U87 and U-251 cells were transduced with Ad.5-IL-24S/IL-15 (5000 vp/cell) or Ad.5-IL-24S, Ad.5 IL-15 or an empty vector control. 72 hours post-transduction, cells were lysed and immunoblotted for PARP cleavage and expression of GRP78. The results showed that the IL-24S/IL-15 fusion protein retained the biological function of IL-24S (FIG. 6).

### Example 5. In Vivo Administration of Ad.5-IL-24S/IL-15 in a GBM Model

This example illustrates the in vivo administration of Ad.5-IL-24S/IL-15 in a GBM mouse model.

To determine the antitumor activity of Ad.5-IL-24S/IL-15 in GL261-Luciferase (Luc), a mouse reporter glioblastoma cell line, cells were injected intracranially to form tumors in the brains of syngeneic C57BL/6 mice (immunocompetent animals). The mice were then subjected to the focused ultrasound-double microbubble (FUS-DMB) approach described herein to evaluate systemic delivery of the Ads (Ad.5 encoding control plasmid (Ad.5 null) or Ad.5-IL-24S, Ad.5 IL-15, Ad.5-IL-24S/IL-15 (also referred to as Ad.5-FSK) or co-transduction of Ad.5-IL-24S+Ad.5 IL-15). This strategy involved systemic delivery of empty microbubbles (MBs) followed by focused ultrasound (FUS) in the brain and then systemic injection of the fusion protein Ad (Ad.5-IL-24S/IL-15; 109 virus particles) encapsulated in MBs followed by FUS in the brain. FUS-DMB first transiently opened the blood brain barrier using empty MBs and FUS, which was rapidly followed by delivering the therapeutic encapsulated in MBs and released in the brain using FUS. For comparison, control empty vector containing Ads were administered using the FUS-DMB approach.

In particular, the FUS-DMB method entailed perfluorocarbon MBs reconstituted in 1 mL of phosphate buffer solution (PBS) containing 108 viral particles of the indicated adenovirus and incubated at 4° C. for 2 hours. After incubation, unenclosed surface-associated Ads were treated with 20% fetal bovine serum (FBS) for 3 hours at 4° C., and any unbound adenovirus were removed by repeated washings. MBs/Ads were then dissolved in 1 mL of PBS before administration. Complement-treated MBs and Ads were systemically injected and sonoporated (using focused ultrasound) e.g., the ultrasound targeted microbubble destruction (UTMD) approach. Sonication was carried out using the FUS transducer as soon as bubble administration was completed (15 seconds). The transducer was driven by a function generator through a power amplifier. A cone filled with degassed and distilled water was attached to the transducer system. Mice were injected with 100 mL of diluted MBs immediately after injection FUS was applied (3.5 mV, 10 dB, 1 MHz). 100 mL of diluted MBs were injected through the tail vein and allowed to circulate for 15 seconds. After 15 seconds, mice were sonicated using FUS for 1 minute as described above. Next the mice were injected intravenously (IV) with 100 mL of MBs containing different Ads and sonicated using FUS for 1 minute after allowing the bubbles to circulate for 15 seconds.

To evaluate the safety profile of the Ad.5 FSK, an in vivo safety assay was performed. Since cytokine release syndrome (CRS) (e.g., cytokine storm) is one of the major complications and causes of toxicity observed following cytokine therapy, the levels of 32 cytokines in the sera of the mice treated were measured using a Quantibody® mouse inflammation array. The levels of cytokines including IL-6, IL-10, IFN-γ, TNFα, VEGF, IL-1β, IL-1α, IL-17, MCP-1, IL-4, IL-2, IL-12(p40), IL-12(p70), and GM-CSF showed no significant difference among the four treatment groups. Meanwhile, Ad.5 null, Ad.5 IL-24S, and Ad.5 FSK, and to an even greater extent Ad.5 IL-15, elevated the levels of lipopolysaccharide-induced CXC chemokine (LIX), a murine neutrophil-chemoattractant, suggesting an ability of the adenovirus-encoded constructs to recruit other cellular components to the tumor site. Small changes in RANTES (chemoattractant), IP-10 (chemokine), and eotaxin (chemoattractant) were also observed in Ad.5 IL-24S and Ad.5 IL-15 groups, as compared to the Ad.5 null group. No robust inflammatory response was observed in any of the treatment groups, indicating that it was unlikely that Ad.5 IL-15, Ad.5 IL-24S, or Ad.5 FSK would induce CRS in vivo.

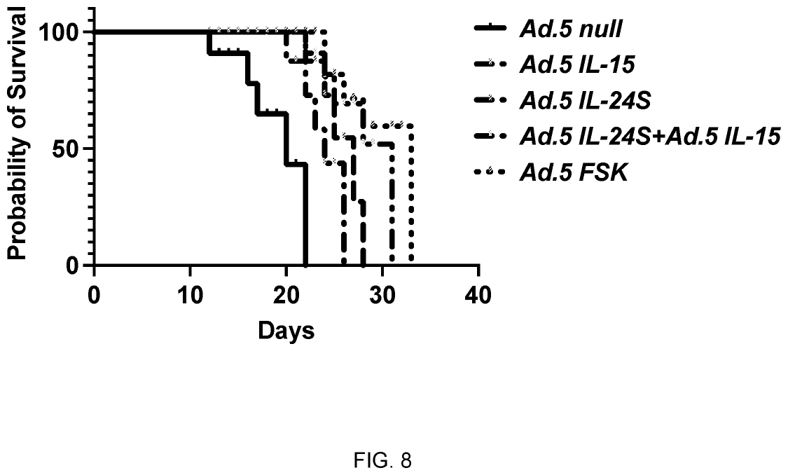

Expression of IL-24S, IL-15, and FSK were observed in brain tumor tissue sections (data not shown). Mice infected with Ad.5 null showed rapid progression of the tumor and died within 30 days, while a single administration of Ad.5 IL-24S or Ad.5 IL-15 using the FUS-DMB approach moderately inhibited GBM progression while infection with Ad.5 FSK had a more pronounced effect in prolonging survival (FIG. 8 and Table 10).

TABLE 10
Statistics of Kaplan-Meier survival curve

| | Log-rank (mantel-cox) Test | | | | |
|---|---|---|---|---|---|
| | Ad.5 | Ad.5 | Ad.5 | Ad.5 IL-24S + | Ad.5 |
| | null | IL-15 | IL-24S | Ad.5 IL-15 | FSK |
| Ad.5 null | | 0.0094 | 0.0002 | 0.0001 | 0.0001 |
| Ad.5 IL-15 | 0.0094 | | 0.69 | .113 | 0.0068 |
| Ad.5 IL-24S | 0.0002 | 0.15 | | .745 | 0.1525 |
| Ad.5 IL-24S + | 0.0001 | .113 | .745 | | .772 |
| Ad.5 IL-15 | | | | | |
| Ad.5 FSK | 0.0001 | 0.0068 | 0.0202 | .1525 | |

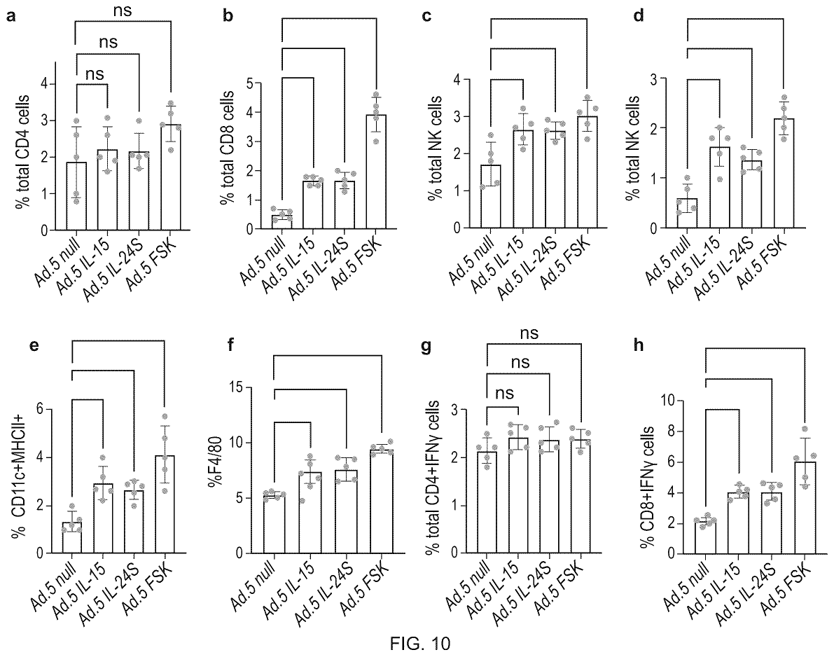

To determine the effects of the FSK in the immunocompetent animals, the percentage of immune cells in tumor tissues were evaluated. C57BL/6 mice were injected intracranially with GL261-Luc cells and treated with equal doses (about 108 vp) of Ad.5 IL-24S, Ad.5 IL-15, Ad.5 FSK, and Ad.5 null in MBs using the FUS-DMB method. Fourteen days after tumor inoculation, the tumors were harvested. Single-cell suspensions of tumor tissues were analyzed for immune cells by flow cytometry for CD4⁺ T cells, CD8⁺ T cells, effector CD4+ (IFN-γ), effector CD8⁺ (IFN-γ), CD3⁻NK1.1⁺ NK cells, CD3⁺ NK1.1⁺ NK cells, and NKT cells. As shown in FIG. 10, Ad.5 IL-24S increased tumor infiltration of CD8⁺ T cells, CD4⁺ T cells, and NK cells, while Ad.5 IL-15 increased the percentage of CD8⁺ T cells, but not CD4⁺ T cells. It was also observed that Ad.5 FSK significantly increased the percentage of both CD4⁺ T cells, CD8⁺ T cells, and NK cells. Additionally, immune enhancement of the percentages of dendritic cells and macrophages were also evident in Ad.5 IL-15- and Ad.5 IL-24S-treated GBM tumors, and Ad.5 FSK further enhanced the percentages of tumor-associated dendritic cells and macrophages. In summary, Ad.5 IL-24S and IL-15 caused significant changes in the immune profile of GBM tumors in the brain and Ad.5 FSK further augmented these immune changes, including enhanced infiltration and recruitment of immune cells in GBM tumors in C57BL/6 mice. The results showed a significant upregulation of multiple diversified immune cells, including cytotoxic T and NK cells in the tumor site resulting from treatment with the fusion protein. This effect was greater than that observed following a single cytokine (IL-24S or IL-15) treatment. Considering survival gains and the enhancement of immune infiltration, these results confirmed the antitumor and immune-modulatory potential of the IL-24S/IL-15 fusion protein.

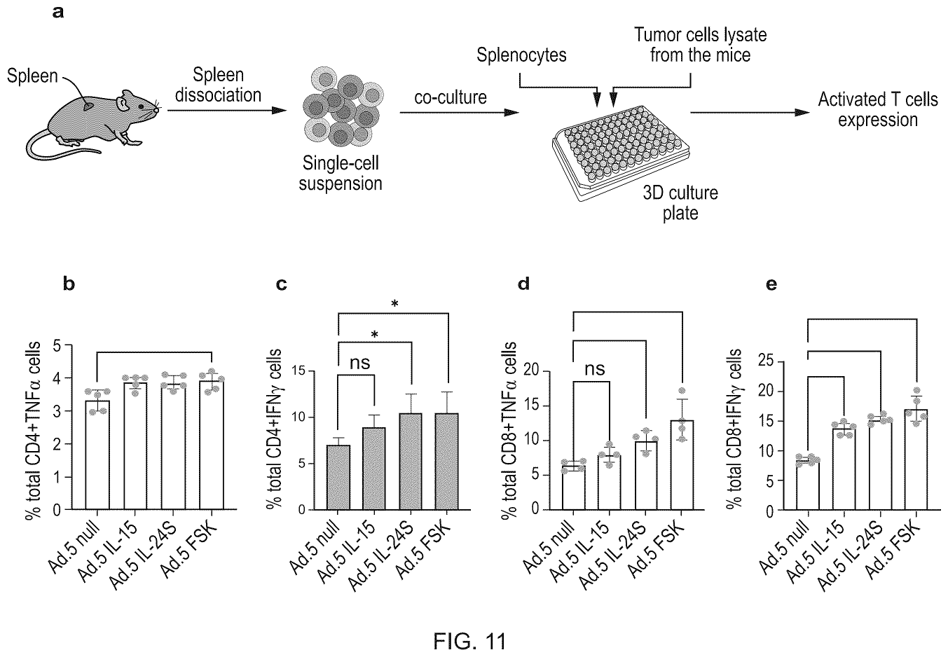

In another set of experiments, C57BL/6 mice were injected intracranially with GL261-Luc cancer cells and treated with equal virus particle number (about 108 VP) of Ad.5 FSK, Ad.5 IL-24S or Ad.5 IL-15. Twenty-two days after tumor inoculation, splenocytes were isolated from these mice and cocultured with irradiated GL261-Luc cells at 37° C. for 2 days. CD4⁺ and CD8⁺ T cells that produced IFNγ or TNFα were analyzed by flow cytometry. As indicated by flow cytometry, treatment with Ad.5 IL-24S, Ad.5 IL-15, and Ad.5 FSK induced an anti-tumor immune response, including induction of tumor-specific cytotoxic T cells (CTLs) and increased NK cell activity. To determine whether a tumor-specific immunological response was generated, the percentage of IFNγ- and TNFα-producing CD4-positive and CD8-positive T cells from the spleens of the cytokine-treated tumor-bearing mice after in vitro stimulation with homologous tumor cells were evaluated (FIG. 11, panel A). As shown in FIG. 11, panels B-E, the percentages of IFNγ-producing CD4-positive and CD8-positive T cells among the cultured splenocytes from Ad.5 IL-24S-treated tumors (FIG. 11, panel C and FIG. 11, panel E) showed a 2.1-fold and 2.3-fold enhancement, respectively. Cultured splenocytes from Ad.5 IL-15-exposed mice showed no significant change in IFNγ-producing CD4⁺ cells but there was almost a 2.1-fold increase in IFNγ-producing CD8-positive cells vs. null-treated mice after stimulation with GL261-Luc cells. The percentages of IFNγ-producing CD4-positive and CD8-positive T cells among the cultured splenocytes from Ad.5 FSK-treated mice were elevated 2- and 2.3-fold, respectively (FIG. 11, panel C and panel E), as compared to the null-treated group. In the case of TNFα-producing immune cells, a similar statistical change was observed in total CD4⁺ cells with all three therapeutic viruses (FIG. 11, panel B), with significant enhancement in CD8⁺ cells evident in Ad.5 IL-24S- and Ad.5 FSK-treated tumors with Ad.5 FSK causing the most pronounced effect (FIG. 11, panel D). Taken together, these results demonstrated that Ad.5 FSK treatment promoted a systemic immunological response to tumor-specific antigens in vivo.

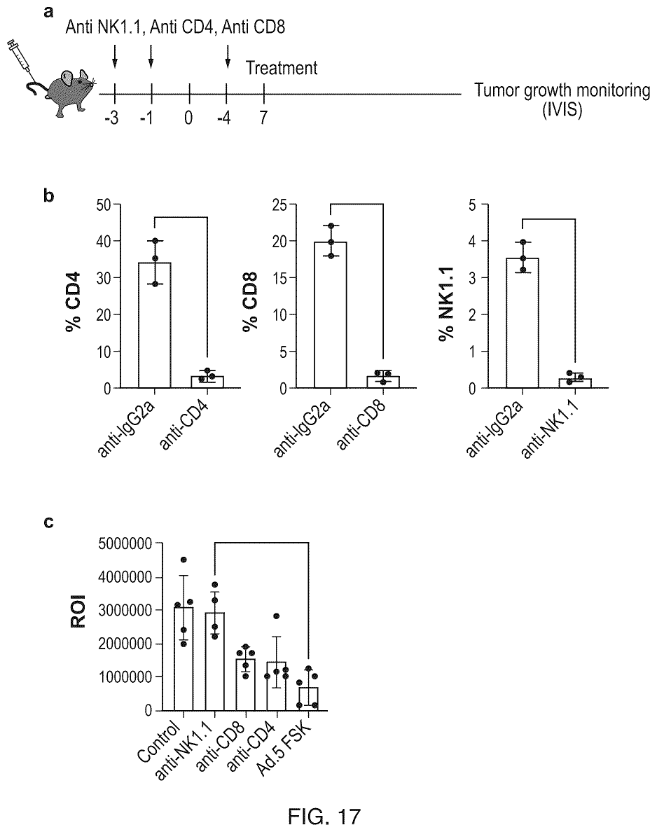

A further mechanistic study demonstrated that NK cells are key effector cells mediating the activity of Ad.5 FSK. In this study, antibody-mediated depletion of CD4+T, CD8+T, and NK cells were conducted to identify critical immune cells contributing to therapeutic responses of the FSK in vivo. A schematic of the experimental design is shown in FIG. 17, panel A. Cellular depletions of CD8⁺ T cells, CD4⁺ T cells, and NK cells were confirmed by flow cytometry of peripheral blood mononuclear cells (PBMCs; FIG. 17, panel B). Depletion of CD8⁺ and CD4⁺ T cells modestly (40% in both cell populations) suppressed the ability of Ad.5 FSK to inhibit GBM growth, as determined by biolayer interferometry (BLI) and In Vivo Imaging System (IVIS)-mediated measurement of the tumor (region of interest (ROI)) size. Importantly, it was observed that NK depletion eliminated Ad.5 FSK suppression of tumor growth (similar to control; FIG. 17, panel C). These results confirmed the role of NK cells in mediating the therapeutic activity of the FSK against GBM.

### Example 6. Sustained Activation and Enhanced In Vitro Cytotoxicity of NK-92 Cells Genetically Engineered to Express IL-24S/IL-15 (FSK)

This example illustrates the sustained activation of genetically engineered NK-92 cells expressing IL-24S/IL-15 (also referred to as FSK) by IL-24S/IL-15 in vitro, which is desired since it is not therapeutically feasible to prime NK cells exogenously or to stimulate them continuously. In the absence of continued in vivo stimulation, ‘cytokine-addicted’ NK cells generally display decreased persistence. This example also illustrates the enhanced cytotoxicity of the genetically engineered NK-92 cells expressing IL-24S/IL-15 on pancreatic ductal adenocarcinoma (PDAC) and prostate cancer cells in vitro.

### Materials and Methods

### Cell Lines

An array of human PDAC cell lines were used, including: MIA PaCa2, AsPC1, PANC-1, BxPC3, and murine PDAC cell lines derived from KPC mice (LSL-Kras^{G12D}; Trp53^{fl/+} or Trp53^{fl/fl}, Pdx-1-Cre).

A total of six castration resistant metastatic human prostate cancer cell lines were used, including: PC-3, PC-3ML, DU-145, ARCaP-M, LNCaP, 22Rv1, and C4-2B; and three murine cell lines Hi-Myc, C2-TRAMP, RM1, and its bone metastatic variant RM1-BM.

### Genetically Engineered Mouse Models (GEMMs)

Besides KRAS, the cardinal tumor suppressors found altered in human PDAC cell lines include TP53, SMAD4, and CDKN2A. To broaden the physiological relevance of the study, mice harboring pancreas-specific homozygous deletion of Trp53 (KPC), Smad4 (KSC), or CDKN2A (KIC) in the background of Kras^{G12D 75-79} were used.

Expansion and Activation of NK Cells from Peripheral Blood Mononuclear Cells (PBMCs) for In Vitro Studies

Cryo-preserved PBMCs were purchased from Lonza Bioscience (Catalog #: CC-2702). NK cells from PBMCs were isolated following conventional protocol/kits (e.g., available from Miltenyi Biotec Corporation).

### Results

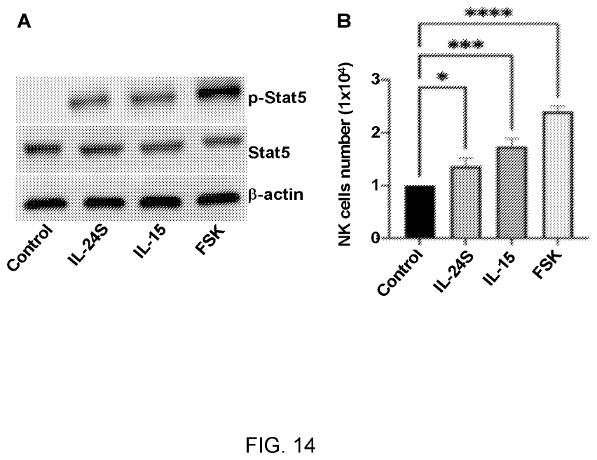

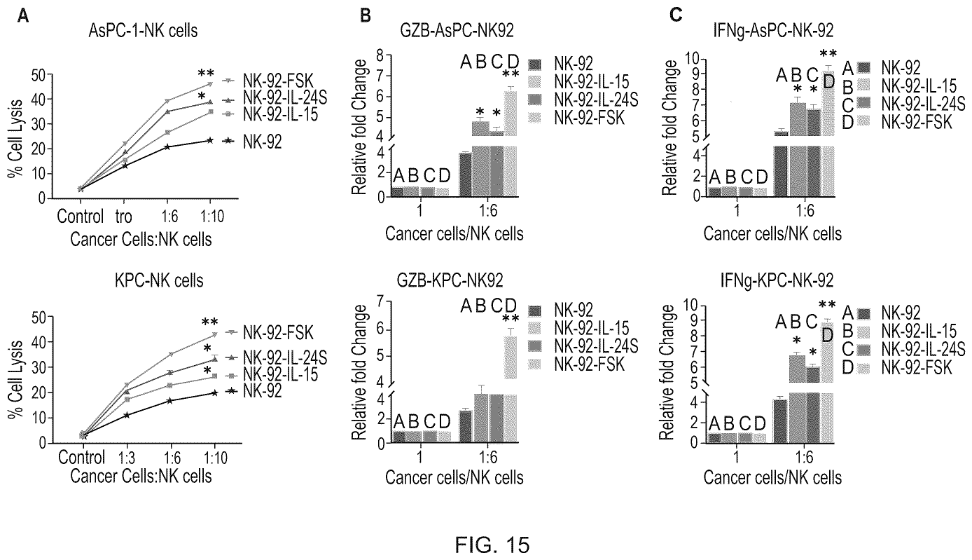

NK-92 cells were genetically engineered to express FSK (NK-92-FSK), IL-15, or IL-24S (NK-92^{IL-24S}), and IL-24S expression and secretion from the engineered cells was confirmed using Western blotting and ELISA (data not shown). In the genetically engineered NK-92 cells, it was observed that NK cell activation markers, including STAT5 (FIG. 14, panel A), secretion of IFNγ (also referred to as INFγ, INF-γ, INF-γ, INFg, and INF-g; FIG. 15, panel C), and activation signature marker GZB (FIG. 15, panel B) exhibited sustained expression. The genetically engineered NK-92 cells expressing FSK also displayed enhanced in vitro expansion (FIG. 14, panel B) compared to NK-92 cells genetically engineered to express IL-24S or IL-15 alone.

To test the cytotoxicity of the genetically engineered NK-92 cells (effector cells), they were incubated with 2×10³ ASPC1 or KPC cells (target cells) at target: NK cell ratios of 1:3, 1:6, and 1:10 in triplicate wells of 96-well plates. It was observed that the NK-92 cells genetically engineered to express FSK exhibited greater cytolytic activity against the two pancreatic ductal adenocarcinoma (PDAC) cells in vitro (FIG. 15).

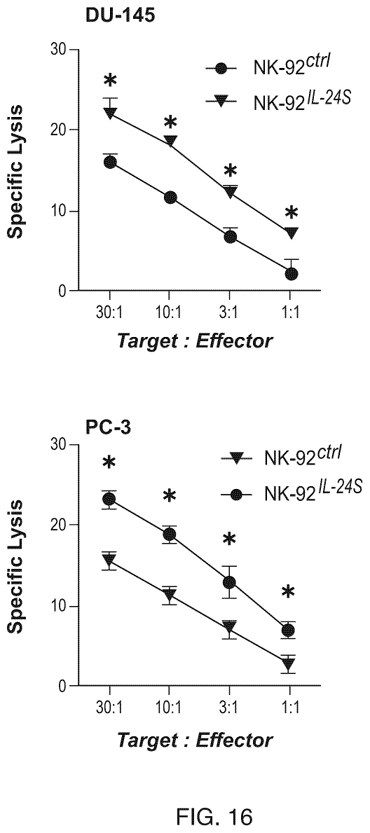

When similar experiments to test the cytotoxicity of the genetically engineered NK-92 cells (effector cells) were conducted with prostate cancer cells (target cells) at target: NK cell ratios of 1:1, 1:3, 1:10, and 1:30, it was observed that the NK-92 cells genetically engineered to express FSK exhibited greater cytolytic activity against two castration-resistant prostate cancer (CRPC) cell lines, DU-145 and PC-3, as compared to parental NK-92 cells (FIG. 16).

### Example 7. Enhanced In Vivo Efficacy of NK-92 Cells Genetically Engineered to Express IL-24S/IL-15 (FSK)

This example illustrates the enhanced cytotoxicity of the genetically engineered NK-92 cells expressing IL-24S/IL-15 on pancreatic ductal adenocarcinoma (PDAC) cells in vivo.

NK-92 cells were genetically engineered to express FSK (NK-92-FSK) or IL-24S, as described in Example 6. Using similar approaches, NK-92 cells were genetically engineered to express IL-15 (NK-IL-15) as well. See Example 6 for additional Materials and Methods.

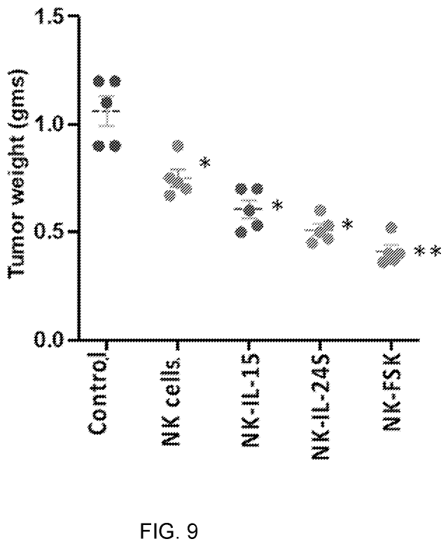

Pancreatic patient-derived xenografts (PDX) were surgically implanted in NOD-scid IL2Rgamma^{null} (NSG) mice. One week later, mice were randomized into 5 groups (n=5). Animals were either untreated or treated with NK cells alone or treated with NK-92-FSK, NK-92^{IL-24S}, or NK-IL-15 (three doses). Once the tumors reached the maximum allowable size, mice were euthanized, and tumors were collected. Tumor weights were measured, and it was observed that mice treated with NK-92-FSK had the smallest tumors (FIG. 9). By a log-rank (Mantel-Cox) test, the tumor weights in the mice treated with NK-92-FSK were significantly lower than the tumor weights in the mice of all other experimental groups, by a p value lower than 0.05.

### Example 8. Enhanced In Vitro Cytotoxicity of NK-92 Cells Genetically Engineered to Express IL-24S/IL-15 (FSK) in Combination with an Antibody that Binds Soluble MICA or Soluble MICB

This example illustrates the enhanced cytotoxicity of the combination of an antibody that binds soluble MICA or soluble MICB in combination with genetically engineered NK-92 cells expressing IL-24S/IL-15 on pancreatic ductal adenocarcinoma (PDAC) cells in vitro.

### Materials and Methods

NK-92 cells were genetically engineered to express FSK (NK-92-FSK), as described in Example 6. See Example 6 for additional Materials and Methods.

### Results

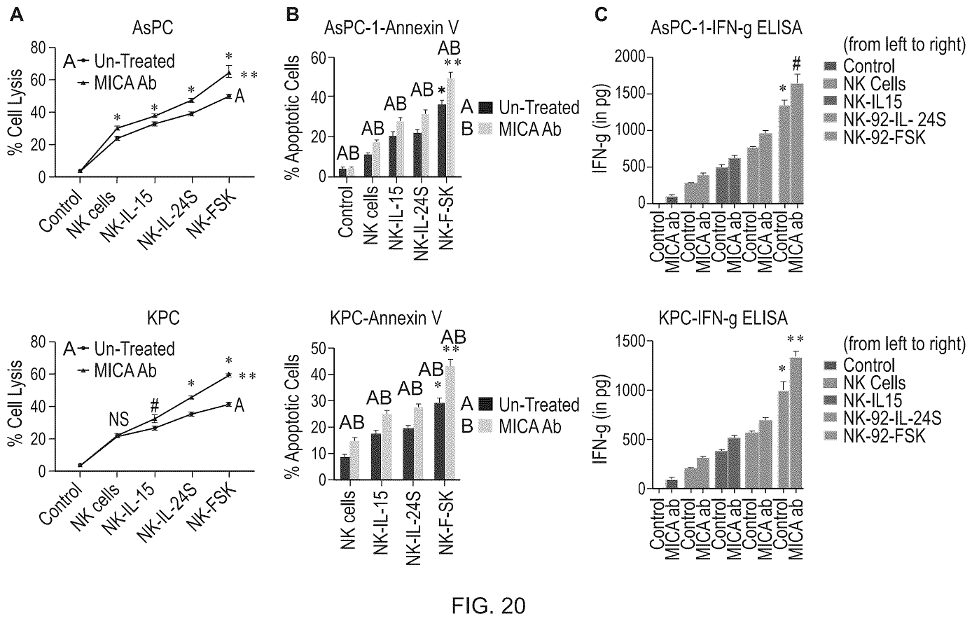

To investigate the anti-tumor activity of NK-92-FSK alone or in combination with an antibody that binds soluble MICA, PDAC cells (AsPC-1 and KPC) were treated with an anti-MICA antibody (1 μg/mL) for 24 hours. NK cells (effector cells) were incubated with 104 AsPC-1 or KPC cells (target cells) with or without antibody treatment at an effector-to-target cell ratio of 3:1 at 37° C. for 24 hours. In result, it was observed that the percentage of target cells that underwent selective lysis by the NK-92-FSK cells was greater in the group treated with the anti-MICA antibody (Ab; FIG. 20, panel A). To quantify cell death, the cells were stained with Annexin V and florescence activated cell sorting (FACS) was performed, which demonstrated that the percentage of apoptotic cells was elevated in the group treated with the anti-MICA antibody (FIG. 20, panel B). Furthermore, the levels of INFg, a marker of NK cell activation, was quantified, and it was observed that the level of INFg was highest in the group treated with the anti-MICA antibody (FIG. 20, panel C).

### Example 9. Enhanced In Vitro Cytotoxicity of NK-92 Cells Genetically Engineered to Express an Anti-PSMA CAR and IL-24S/IL-15

This example illustrates the construction of genetically engineered PSMA CAR NK-92 cells expressing IL-24S/IL-15 or expressing IL-24S, as well as the enhanced cytotoxicity of the genetically engineered PSMA CAR NK-92 cells expressing IL-24S on prostate cancer cells in vitro.

### Materials and Methods

### Cell Lines

A total of six castration-resistant metastatic human prostate cancer cell lines were used, including: PC-3, PC-3ML, DU-145, ARCaP-M, LNCaP, 22Rv1, and C4-2B; and three murine cell lines Hi-Myc, C2-TRAMP, RM1, and its bone metastatic variant RM1-BM.

Expansion and Activation of NK Cells from Peripheral Blood Mononuclear Cells (PBMCs) for In Vitro Studies

Cryo-preserved PBMCs were purchased from Lonza Bioscience (Catalog #: CC-2702). NK cells from PBMCs were isolated following conventional protocol/kits (e.g., available from Miltenyi Biotec Corporation).

Designing ^{PSMA}NK-92-FSK and ^{PSMA}NK-92^{IL-24S}

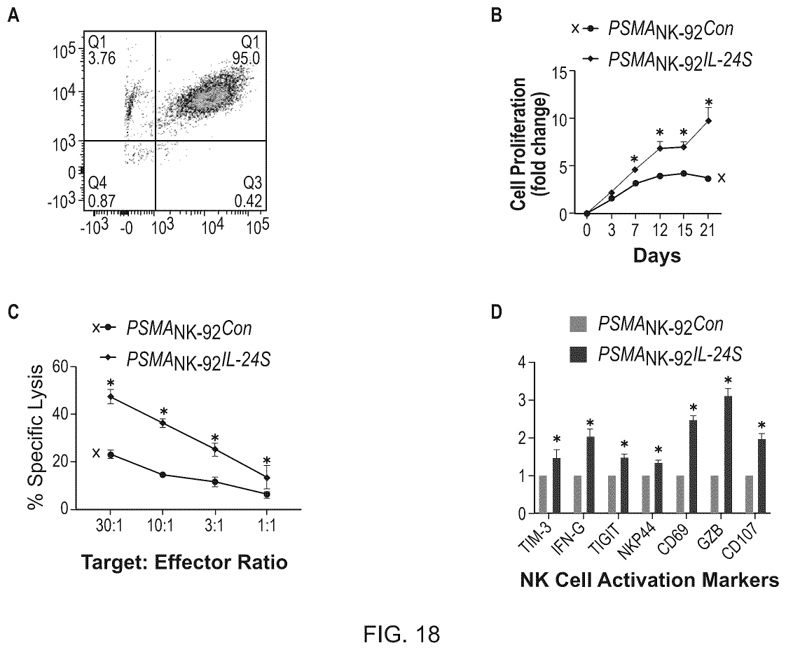

Unmodified NK cells lack precision targeting when used conventionally. To further enhance the specificity and target selectivity of genetically engineered NK-92 cells expressing FSK or IL-24S, prostate-specific membrane antigen (PSMA) chimeric antigen receptor (CAR)-engineered NK-92 cells were generated (^{PSMA}NK-92-FSK) and ^{PSMA}NK-92^{IL 24S}, respectively), which, via the PSMA CAR, targeted both PDAC cells, as well as prostate cancer cells. Briefly, NK-92 cells were transduced with a lentiviral vector encoding PSMA CAR-IL-24S (or control; ^{PSMA}NK-92^{Con}), composed of an extracellular scFv antibody linked through a hinge region to a mouse CD8 transmembrane region and a mouse CD28 signaling domain and CD3-zeta chain. The transduction efficiency of PSMA CAR-IL-24S in NK-92 cells were determined by flow cytometry, and it was demonstrated that the ^{PSMA}NK-92^{IL-24S} cells expressed PSMA (FIG. 18, panel A). In additional control experiments, ^{PSMA}NK-92^{IL-24S} cells were co-cultured with PC-3^{PSMA} and the activation of specific NK-92 activation markers were confirmed (FIG. 18, panel D).

### Results

The genetically engineered ^{PSMA}NK-92^{IL-24S} cells displayed enhanced in vitro expansion compared to ^{PSMA}NK-92 cells not expressing IL-24S (FIG. 18, panel B).

To test the cytotoxicity of the ^{PSMA}NK-92^{IL-24S} cells (effector cells), they were incubated with 2×10³ PSMA-overexpressing PC-3 cells (PC-3^{PSMA}; target cells) at ratios of 1:1, 1:3, 1:10, and 1:30 in triplicate wells of 96-well plates in a standard 4 hour lactate dehydrogenase (LDH) release assay. It was observed that the PC-3^{PSMA} cells greatly outperformed the PC-3 cells in terms of the percentage of target cells that underwent selective lysis by the ^{PSMA}NK-92^{IL-24S} cells (FIG. 18, panel C).

### Example 10. Enhanced In Vivo Efficacy of NK-92 Cells Genetically Engineered to Express IL-24S and an Anti-PSMA CAR

This example illustrates the cytotoxic efficacy of genetically engineered PSMA CAR NK-92 cells expressing IL-24S in a PC-3^{PSMA} xenograft model in vivo.

### Materials and Methods

^{PSMA}NK-92^{IL-24S} cells were designed, as described in Example 9.

### Xenograft Model

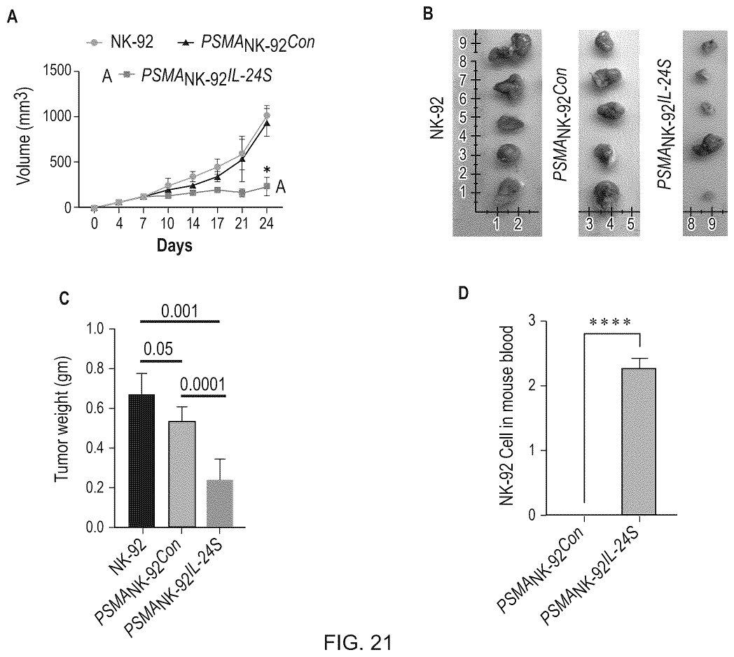

106 PC-3^{PSMA} cells (described in Example 9), were implanted in the right flank of mice to develop xenografts. Once a tumor became palpable, mice were randomly assigned into three experimental groups: control NK-92 cells, ^{PSMA}NK-92^{Con} (described in Example 9), and ^{PSMA}NK-92^{IL-24S} (described in Example 9) and were administered the respective NK-92 cells on experimental days 4, 7, and 10. Using caliper measurements, tumor sizes (FIG. 21, panel A) and weights (FIG. 21, panel C) were estimated and recorded. Tumor size was also visualized with photomicrographs (FIG. 21, panel B).

### Results

Mice treated with ^{PSMA}NK-92^{IL-24S} had significantly smaller tumors (FIG. 21, panels A-C). Additionally, ^{PSMA}NK-92^{IL-24S} but not ^{PSMA}NK-92^{Con} cells were recovered 10 days after infusion (FIG. 21, panel D), demonstrating that that IL-24S arming positively impacts in vivo persistence of therapeutic NK cells.

### Example 11. IL-24S Administration is Safe In Vivo

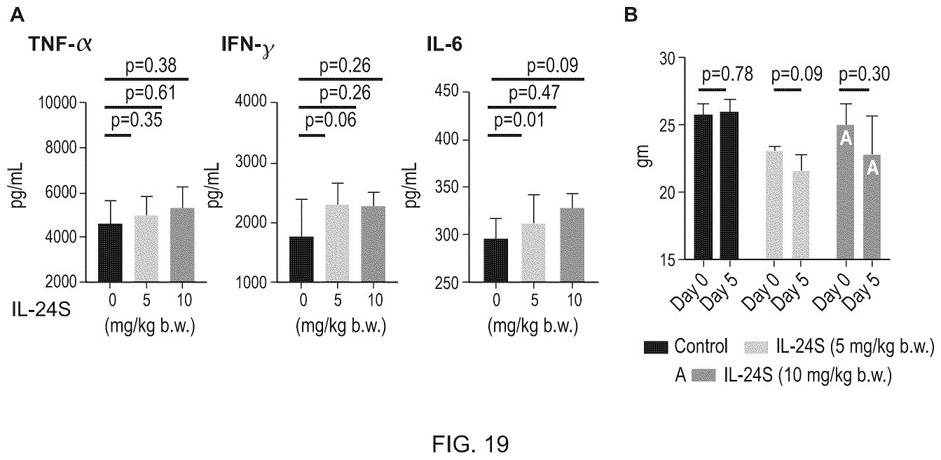

The immune system of NSG mice were humanized using fetal cord blood CD34⁺ progenitors. It was observed that the administration of IL-2 recapitulated in vivo pathological changes and human-specific immune responses, e.g., elevated cytokine levels (IL-6 and IFNg) and modulation of CD4 and NK cells, confirming model authenticity with 18% body weight loss within a week (an indication of a ‘cytokine storm’ with potential multi-organ failure; data not shown). While IL-2 demonstrated toxicity, when IL-24S was administered intravenously, no changes in TNF-α, IFNg, or IL-6 (FIG. 19, panel A) or body weight (FIG. 19, panel B) in the mice were observed, demonstrating the safety of IL-24S.

### INCORPORATION BY REFERENCE

All publications and patents cited throughout the specification and claims herein (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

What is claimed is:

1. A fusion protein comprising an IL-24 polypeptide and an immunostimulatory cytokine.

2. The fusion protein of claim 1, wherein the immunostimulatory cytokine comprises an IL-15 polypeptide.

3. The fusion protein of claim 2, wherein the IL-15 polypeptide comprises an amino acid sequence at least 85%, at least 90%, at least 95%, or at least 99% identical to the amino acid sequence of SEQ ID NO: 14.

4. The fusion protein of claim 3, wherein the IL-15 polypeptide comprises the amino acid sequence of SEQ ID NO: 14.

5. The fusion protein of any one of claims 2-4, wherein the C-terminus of the IL-24 polypeptide is fused to the N-terminus of the IL-15 polypeptide.

6. The fusion protein of any one of claims 2-5, wherein the IL-24 polypeptide is fused to the IL-15 polypeptide via a linker.

7. The fusion protein of claim 6, wherein the linker comprises the amino acid sequence of SEQ ID NO: 33.

8. The fusion protein of claim 6 or 7, wherein the amino acid sequence of the fusion protein comprises the amino acid sequence of SEQ ID NO: 41.

9. The fusion protein of claim 1, wherein the immunostimulatory cytokine comprises an IL-12 polypeptide.

10. The fusion protein of claim 1, wherein the immunostimulatory cytokine comprises an IL-2 polypeptide.

11. The fusion protein of any one of claims 1-10, wherein the immunostimulatory cytokine stimulates an NK cell.

12. The fusion protein of any one of claims 1-11, wherein the immunostimulatory cytokine stimulates a T cell.

13. The fusion protein of any one of claims 1-12, wherein the IL-24 polypeptide comprises an amino acid sequence at least 90% identical to SEQ ID NO: 5.

14. The fusion protein of claim 13, wherein the IL-24 polypeptide comprises a mutation corresponding to K19R relative to SEQ ID NO: 5.

15. The fusion protein of any one of claims 1-[0080], wherein the IL-24 polypeptide comprises the amino acid sequence of SEQ ID NO: 6.

16. The fusion protein of any one of claims 1-[0081], wherein the IL-24 polypeptide comprises an amino acid sequence at least 90% identical to SEQ ID NO: 3.

17. The fusion protein of claim 16, wherein the IL-24 polypeptide comprises the amino acid sequence of SEQ ID NO: 4.

18. The fusion protein of any one of claims 1-17, wherein the IL-24 polypeptide does not comprise amino acids 1-51 of SEQ ID NO: 1.

19. A nucleic acid encoding the fusion protein of any one of claims 1-18.

20. The nucleic acid of claim 19, further encoding an N-terminal signal peptide.

21. The nucleic acid of claim 20, wherein the signal peptide is an Insulin signal peptide.

22. The nucleic acid of claim 21, wherein the Insulin signal peptide is a human Insulin signal peptide comprising an amino acid sequence at least 90% identical to the amino acid sequence of SEQ ID NO: 8.

23. The nucleic acid of claim 22, wherein the Insulin signal peptide comprises the amino acid sequence of SEQ ID NO: 8.

24. The nucleic acid of any one of claims 19-23, wherein the nucleic acid comprises a nucleotide sequence at least 90% identical to SEQ ID NO: 12.

25. The nucleic acid of claim 24, wherein the nucleic acid comprises the nucleotide sequence of SEQ ID NO: 12.

26. A vector comprising the nucleic acid of any one of claims 19-25.

27. The vector of claim 26, wherein the vector is a viral vector.

28. The vector of claim 27, wherein the viral vector is an adenovirus vector.

29. The vector of claim 28, wherein the adenovirus vector comprises a cancer-selective promoter operably linked to E1A and/or E1B genes.

30. The vector of claim 29, wherein the cancer-selective promoter comprises a PEG-3 promoter or a pGAPE promoter.

31. The vector of any one of claims 28-30, wherein the adenovirus vector comprises a chimeric serotype 5/3 adenovirus fiber protein that comprises tail and shaft regions of an Ad.5 fiber protein and a knob region of an Ad.3 fiber protein.

32. A cell comprising the nucleic acid of any one of claims 19-25 or the vector of any one of claims 26-31.

33. The cell of claim 32, wherein the cell is an NK cell.

34. The cell of claim 32, wherein the cell is a T cell.

35. An NK cell comprising a nucleic acid encoding an IL-24 polypeptide operably linked to a heterologous transcription control element.

36. The NK cell of claim 35, wherein the IL-24 polypeptide comprises an amino acid sequence at least 90% identical to SEQ ID NO: 5.

37. The NK cell of claim 36, wherein the IL-24 polypeptide comprises a mutation corresponding to K19R relative to SEQ ID NO: 5.

38. The NK cell of any one of claims 35-37, wherein the IL-24 polypeptide comprises the amino acid sequence of SEQ ID NO: 6.

39. The NK cell of any one of claims 35-38, wherein the IL-24 polypeptide comprises an amino acid sequence at least 90% identical to SEQ ID NO: 3.

40. The NK cell of claim 39, wherein the IL-24 polypeptide comprises the amino acid sequence of SEQ ID NO: 4.

41. The NK cell of any one of claims 35-40, wherein the IL-24 polypeptide does not comprise amino acids 1-51 of SEQ ID NO: 1.

42. The NK cell of claim 41, wherein the nucleic acid further encodes an N-terminal signal peptide.

43. The NK cell of claim 42, wherein the signal peptide is an Insulin signal peptide.

44. The NK cell of claim 43, wherein the Insulin signal peptide is a human Insulin signal peptide comprising an amino acid sequence at least 90% identical to the amino acid sequence of SEQ ID NO: 8.

45. The NK cell of claim 44, wherein the Insulin signal peptide comprises the amino acid sequence of SEQ ID NO: 8.

46. The NK cell of any one of claims 35-45, wherein the nucleic acid comprises a nucleotide sequence at least 90% identical to SEQ ID NO: 12.

47. The NK cell of claim 46, wherein the nucleic acid comprises the nucleotide sequence of SEQ ID NO: 12.

48. The cell of any one of claims 32-47, wherein the cell secretes the IL-24 polypeptide or the fusion protein.

49. The cell of any one of claims 32-48, wherein the cell further comprises an antigen-specific receptor that binds a tumor-associated antigen.

50. The cell of claim 49, wherein the antigen-specific receptor comprises a chimeric antigen receptor (CAR).

51. A pharmaceutical composition comprising:
(a) the fusion protein of any one of claims 1-18, the nucleic acid of any one of claims 19-25, or the vector of any one of claims 26-31; and
(b) a pharmaceutically acceptable carrier or excipient.

52. A method of treating cancer, the method comprising: administering to a subject in need thereof a therapeutically effective amount of the pharmaceutical composition of claim 51.

53. The method of claim 52, wherein the cancer is a solid tumor.

54. The method of claim 53, wherein the solid tumor is a glioblastoma, prostate cancer, melanoma, liver cancer, prostate cancer, kidney cancer, pancreatic cancer, lung cancer, breast cancer, or bone cancer.

55. The method of claim 52, wherein the cancer is a leukemia.

56. The method of claim 53 or 54, wherein the pharmaceutical composition is administered at a site of the solid tumor.

57. The method of claim 56, wherein the pharmaceutical composition comprises the fusion protein, nucleic acid, or vector encapsulated in a microbubble, and wherein the method comprises administering the microbubble systemically and applying focused ultrasound to the site of the solid tumor to disrupt the microbubble.

58. The method of claim 57, wherein the method further comprises, prior to administration of the pharmaceutical composition:
(a) administering to the subject a penetrating microbubble composition that comprises a microbubble not containing the pharmaceutical composition; and
(b) applying focused ultrasound to the site of the solid tumor to disrupt the microbubble in the penetrating microbubble composition.

59. The method of claim 58, wherein the pharmaceutical composition is administered within 4 hours, 2 hours, 1 hour, 30 minutes, 10 minutes, or 5 minutes after administering the penetrating microbubble composition.

60. The method of claim 58 or 59, wherein administering the pharmaceutical composition and the penetrating microbubble composition are administered intravenously.

61. The method of any one of claims 52-55, wherein the pharmaceutical composition is administered systemically.

62. A pharmaceutical composition comprising:
(a) the cell of any one of claims 32-50; and
(b) a pharmaceutically acceptable carrier or excipient.

63. A method of treating cancer, the method comprising: administering to a subject in need thereof a therapeutically effective amount of the pharmaceutical composition of claim 62.

64. The method of claim 63, wherein the pharmaceutical composition is administered by intravenous infusion.

65. The method of any one of claims 52-64, further comprising administering to the subject a therapeutically effective amount of an antibody that binds soluble MICA or soluble MICB.
